(19) United States
(12) Patent Application Publication
(10) Pub. No.: US 2022/0002688 A1
(43) Pub. Date: Jan. 6, 2022

(54) PURIFICATION OF IDURONATE-2-SULFATASE IMMUNOGLOBULIN FUSION PROTEIN

(71) Applicant: Armagen, Inc., San Diego, CA (US)

(72) Inventors: Rahul CHELIKANI, Lexington, MA (US); Hang YUAN, Lexington, MA (US); Francis BACON, Lexington, MA (US); Ying YANG, Lexington, MA (US)

(21) Appl. No.: 17/295,400

(22) PCT Filed: Dec. 20, 2019
(86) PCT No.: PCT/US2019/067868

§ 371 Date: May 19, 2021

Related U.S. Application Data
(60) Provisional application No. 62/782,834, filed on Dec. 20, 2018.

(51) Int. Cl. C12N 9/16; C07K 16/28

(52) U.S. Cl. CPC C12N 9/16; C12Y 301/06013; A61K 38/00; C07K 2319/30; C07K 2317/24; C07K 16/2869

(57) ABSTRACT

The present invention relates to an improved composition comprising purified fusion protein including an immunoglobulin and an iduronate-2-sulfatase (12S). In some embodiments, the fusion protein comprises at least about 60% conversion of the cysteine residue corresponding to Cys59 of SEQ ID NO:1 [wild-type human 12S] to C α-formylglycine (FGly), wherein the purified fusion protein is characterized with between 1% and 10% 2-mannose-6-phosphate (2-M6P) peak area on glycan map.

### CROSS-REFERENCE TO RELATED APPLICATIONS

This application claims benefit of, and priority to U.S. Provisional Patent Application Ser. No. 62/782,834 filed on Dec. 20, 2018, the contents of which are incorporated herein in its entirety.

### INCORPORATION BY REFERENCE OF SEQUENCE LISTING

The content of the text file named “SHR-182-AGT-SequenceListing.txt”, which was created on Dec. 18, 2019 and is 37196 bytes in size, is hereby incorporated by reference in its entirety.

### BACKGROUND

Mucopolysaccharidosis type II (MPS II, Hunter syndrome) is an X-chromosome-linked recessive lysosomal storage disorder that results from a deficiency in the enzyme iduronate-2-sulfatase (I2S). I2S cleaves the terminal 2-O-sulfate moieties from the glycosaminoglycans (GAG) dermatan sulfate and heparan sulfate. Due to the missing or defective I2S enzyme in patients with Hunter syndrome, GAG progressively accumulate in the lysosomes of a variety of cell types, leading to cellular engorgement, organomegaly, tissue destruction, and organ system dysfunction.

Generally, physical manifestations for people with Hunter syndrome include both somatic and neuronal symptoms. For example, in some cases of Hunter syndrome, central nervous system (CNS) involvement leads to developmental delays and nervous system problems. Symptoms such as neurodegeneration and mental retardation appear during childhood, and Hunter syndrome patients suffering from neuronal effects often die at an early age due to organ damage to the brain. Similarly, the accumulation of GAG can adversely affect the organ systems of the body. Manifesting initially as a thickening of the wall of the heart, lungs and airways, and abnormal enlargement of the liver, spleen and kidneys, these profound changes can ultimately lead to widespread catastrophic organ failure. As a result, Hunter syndrome is always severe, progressive, and life-limiting.

Enzyme replacement therapy (ERT) is an approved therapy for treating Hunter syndrome (MPS II), which involves administering exogenous replacement I2S enzyme to patients with Hunter syndrome. However, systemically administered I2S enzyme does not readily cross the blood brain barrier (BBB) and thus often proves insufficient in treating CNS manifestations of the disease.

### SUMMARY

The present invention provides, among other things, highly potent iduronate-2-sulfatase fusion protein that can effectively cross the blood brain barrier (BBB) for treatment of CNS symptoms associated with Hunter syndrome. In addition, the present invention provides improved methods and compositions for treating Hunter syndrome via enzyme replacement therapy. The present invention is, in part, based on the surprising discovery that Human Insulin Receptor Antibody-I2S (HIRMab-I2S) fusion protein can be purified from unprocessed biological materials, such as, HIRMab-I2S fusion protein-containing cell culture medium, using a process involving as few as three chromatography columns. The present invention allows for the modulation of 2-mannose-6-phosphate (2-M6P or bis-M6P) levels that may increase facilitation of bioavailability and/or lysosomal targeting of the I2S enzyme. As described in the Examples section, HIRMab-I2S fusion proteins purified using a three-column process according to the invention retains high percentage of C_{α}-formylglycine (FGly) (e.g., higher than 70% and up to 100%), which is important for the activity of I2S enzyme. In addition, HIRMab-I2S fusion protein purified according to the present invention demonstrate high purity levels (<8 ppm Host Cell Protein). Therefore, the present invention provides a more effective, cheaper, and faster process for purifying HIRMab-I2S fusion protein.

It is understood that any of the aspects and embodiments described below can be combined in any desired way, and that any embodiment or combination of embodiments can be applied to each of the aspects described below, unless the context indicates otherwise.

In one aspect, a composition is provided comprising a purified fusion protein including an immunoglobulin and an iduronate-2-sulfatase (I2S), wherein the fusion protein comprises at least about 60% conversion of the cysteine residue corresponding to Cys59 of SEQ ID NO:1 to Cα-formylglycine (FGly), wherein the purified fusion protein is characterized with between 1% and 10% 2-mannose-6-phosphate (2-M6P) peak area on glycan map. In embodiments, the purified fusion protein is characterized with between 4% and 9% 2-mannose-6-phosphate (2-M6P) peak area on glycan map. In embodiments, the purified fusion protein is characterized with between 5.2% and 7.2% 2-mannose-6-phosphate (2-M6P) peak area on glycan map. In embodiments, the fusion protein comprises at least about 60% conversion of the cysteine residue corresponding to Cys59 of SEQ ID NO:1 to Cα-formylglycine (FGly), wherein the purified fusion protein comprises between, on average, about 3.0 mol/mol and about 4.0 mol/mol mannose-6-phosphate (2-M6P) residues per molecule. In embodiments, the purified fusion protein comprises at least about 70% conversion of the cysteine residue corresponding to Cys59 of SEQ ID NO:1 to Cα-formylglycine (FGly). In embodiments, the purified fusion protein comprises at least about 80% conversion of the cysteine residue corresponding to Cys59 of SEQ ID NO:1 to Cα-formylglycine (FGly). In embodiments, the purified fusion protein comprises at least about 90% conversion of the cysteine residue corresponding to Cys59 of SEQ ID NO:1 to Cα-formylglycine (FGly). In embodiments, the purified fusion protein comprises at least about 95% conversion of the cysteine residue corresponding to Cys59 of SEQ ID NO:1 to Cα-formylglycine (FGly). In embodiments, the purified fusion protein comprises at least about 98% conversion of the cysteine residue corresponding to Cys59 of SEQ ID NO:1 to Cα-formylglycine (FGly). In embodiments, the purified fusion protein is derived from mammalian cells. In embodiments, the purified fusion protein is derived from CHO cells. In embodiments, the purified fusion protein comprises between 5.2% and 7.2% 2-M6P residue levels. In embodiments, the purified fusion protein includes an immunoglobulin comprising a chimeric monoclonal antibody. In embodiments, the immunoglobulin comprises a chimeric monoclonal antibody that binds to Human Insulin Receptor (HIR). In embodiments, the purified fusion protein comprises a human insulin receptor monoclonal antibody fused with I2S. In embodiments, the purified fusion protein comprises an amino acid sequence at least 80%, 85%, 90% or 95% identical to SEQ ID NO: 11. In embodiments, the purified fusion protein comprises an amino acid sequence identical to SEQ ID NO: 11. In embodiments, the chimeric monoclonal antibody comprises a recombinant human IgG light chain. In embodiments, the recombinant human IgG light chain comprises an amino acid sequence at least 80%, 85%, 90% or 95% identical to SEQ ID NO: 12. In embodiments, the recombinant human IgG light chain comprises an amino acid sequence identical to SEQ ID NO: 12. In embodiments, the fusion protein does not comprise a linker. In embodiments, the Human Insulin Receptor mediate transport via endogenous brain capillary endothelial insulin receptors. In embodiments, the Human Insulin Receptor mediate transport via endogenous neuronal insulin receptors. In embodiments, the purified fusion protein includes an iduronate-2-sulfatase comprising 2-M6P residues. In embodiments, the 2-M6P residues bind to M6P receptor. In embodiments, the 2-M6P receptor mediate transport via endogenous lysosomal M6P receptor. In embodiments, the 2-M6P residues facilitate at least about 60% binding to M6P receptor. In embodiments, the 2-M6P residues facilitate at least about 70% binding to M6P receptor. In embodiments, the 2-M6P residues facilitate at least about 75% binding to M6P receptor. In embodiments, the immunoglobulin facilitates at least about 70% binding to Human Insulin Receptor. In embodiments, the immunoglobulin facilitates at least about 80% binding to Human Insulin Receptor. In embodiments, the immunoglobulin facilitates at least about 90% binding to Human Insulin Receptor. In embodiments, the immunoglobulin facilitates at least about 95% binding to Human Insulin Receptor. In embodiments, the purified fusion protein has a specific activity of at least about 3 U/mg as determined by a plate-based fluorometric enzyme assay. In embodiments, the purified fusion protein contains less than 10 ng/mg (ppm) HCP. In embodiments, the purified fusion protein contains at least 15 mol/mol sialic acid content. In embodiments, the purified fusion protein contains at least 20 mol/mol sialic acid content.

In another aspect, a method is provided comprising purifying a fusion protein including an immunoglobulin and an iduronate-2-sulfatase (I2S) from an impure preparation by conducting one or more of affinity chromatography, cation-exchange chromatography, and multimodal chromatography. In embodiments, the affinity chromatography is Protein A Antibody chromatography. In embodiments, the cation-exchange chromatography is Capto SP ImpRes chromatography. In embodiments, the multimodal chromatography is Capto Adhere chromatography. In embodiments, the method involves 3 chromatography steps. In embodiments, the method conducts the affinity chromatography, cation-exchange chromatography, and multimodal chromatography in that order. In embodiments, the affinity chromatography column is eluted using an elution buffer comprising an isocratic sodium citrate elution. In embodiments, the isocratic sodium citrate elution comprises a range from 10-100 mM sodium citrate. In embodiments, the affinity chromatography column is run at a pH of between 3.3 and 3.9. In embodiments, the cation-exchange chromatography column is eluted using an elution buffer comprising an isocratic NaCl elution. In embodiments, the NaCl elution comprises a range from 10-300 mM NaCl. In embodiments, the cation-exchange chromatography column is run at a pH of between 5.2 and 5.8. In embodiments, the multimodal chromatography column is operated in flow through mode and/or bind/elute mode. In embodiments, a salt concentration of between 1.0 and 2.0 M NaCl is used in loading and washing the chromatography columns. In embodiments, the multimodal chromatography column is run at a pH of about 7.0. In embodiments, the method further comprises a step of viral inactivation. In embodiments, the method further comprises a step of vial filtration after the last chromatography column. In embodiments, the fusion protein including an immunoglobulin and an I2S protein is produced by mammalian cells cultured in chemically defined medium. In embodiments, the mammalian cells are CHO cells. In embodiments, the mammalian cells are cultured in a bioreactor. In embodiments, the bioreactor operates as a stirred tank perfusion bioreactor process. In embodiments, the impure preparation is prepared from the chemically defined medium containing fusion protein secreted from the mammalian cells.

In another aspect, a pharmaceutical composition is provided comprising a purified fusion protein including an immunoglobulin and an I2S protein purified according to a method of any one of the preceding claims. In embodiments, the immunoglobulin comprises a chimeric monoclonal antibody that binds to the Human Insulin Receptor (HIR). In embodiments, the purified fusion protein comprises an I2S polypeptide and a chimeric monoclonal antibody that binds to the Human Insulin Receptor (HIR). In embodiments, the purified fusion protein comprises an amino acid sequence at least 80%, 85%, 90% or 95% identical to SEQ ID NO: 11. In embodiments, the purified fusion protein comprises an amino acid sequence identical to SEQ ID NO: 11. In embodiments, the chimeric monoclonal antibody comprises a recombinant human IgG light chain. In embodiments, the recombinant human IgG light chain comprises an amino acid sequence at least 80%, 85%, 90% or 95% identical to SEQ ID NO: 12. In embodiments, the recombinant human IgG light chain comprises an amino acid sequence identical to SEQ ID NO: 12.

In another aspect, a method of treating Hunter syndrome is provided comprising administering to a subject in need of treatment a pharmaceutical composition as described herein.

Other features and advantages of the invention will be apparent from the drawings and the following detailed description and claims.

### BRIEF DESCRIPTION OF THE DRAWINGS

The above and further features will be more clearly appreciated from the following detailed description when taken in conjunction with the accompanying drawings. The drawings however are for illustration purposes only; not for limitation.

FIG. 1 depicts an exemplary purification scheme for HIRMab-I2S fusion protein produced in chemically-defined medium.

FIG. 2 depicts analysis of specific activity (U/mg) and formylglycine content (% FG) of early, mid, and late HIRMab-I2S fusion protein harvest materials obtained under various media conditions, including chemically-defined OptiCHO, chemically-defined OptiCHO with Cell boost 5 additive, and SFM4CHO medium containing hydrolysates and animal components.

FIG. 3 depicts an analysis of results from a Substrate Clearance assay, a binding to the Human insulin receptor assay, and a binding to M6P receptor assay of early and late HIRMab-I2S fusion protein harvest materials obtained under various media conditions, including chemically-defined OptiCHO, chemically-defined OptiCHO with Cell boost 5 additive, and SFM4CHO medium containing hydrolysates and animal components.

FIG. 4 depicts an analysis of the levels of mannose-6-phosphate glycan content (1-M6P and 2-M6P) in early, mid, and late HIRMab-I2S fusion protein harvest materials obtained under various media conditions, including chemically-defined OptiCHO, chemically-defined OptiCHO with Cell boost 5 additive, and SFM4CHO medium containing hydrolysates and animal components.

FIG. 5 depicts an analysis of the levels of sialylation glycan content (1-, 2-, 3-, and 4-SA) in early, mid, and late HIRMab-I2S fusion protein harvest materials obtained under various media conditions, including chemically-defined OptiCHO, chemically-defined OptiCHO with Cell boost 5 additive, and SFM4CHO medium containing hydrolysates and animal components.

FIG. 6 depicts an analysis of the levels of neutral and Peak 8 glycan content in early, mid, and late HIRMab-I2S fusion protein harvest materials obtained under various media conditions, including chemically-defined OptiCHO, chemically-defined OptiCHO with Cell boost 5 additive, and SFM4CHO medium containing hydrolysates and animal components.

FIG. 7 depicts a specific activity assay for HIRMab-I2S. In the first step, the substrate, Ido2S-4-MU is hydrolyzed to IdoA-4-MU and sulfate by I2S. In the second step, 4-MU is released by the action of α-L-iduronidase (IDUA). Fluorescence quantitation of the 4-MU product is carried out following high pH quench.

FIG. 8 depicts the I2S activity of 2.5 ng/mL of HIRMab-I2S in various buffer conditions.

FIG. 9 depicts a least-square fit to the linearized Equation 4 to determine matrix-free activity.

FIG. 10A depicts the activity of HIRMab-I2S in Buffer 3 as a function of serial dilution across the dilution range indicated (DF: dilution factor). FIG. 10B depicts the activity of HIRMab-I2S in Buffer 4 as a function of serial dilution across the dilution range indicated (DF: dilution factor). FIG. 10C depicts the activity of HIRMab-I2S in Buffer 6 as a function of serial dilution across the dilution range indicated (DF: dilution factor).

FIG. 11 depicts a schematic representation of experiment conducted in Buffer 3 to separate the effects of matrix and enzyme concentration on specific activity.

FIG. 12 depicts substrate depletion in the experiment conducted in FIG. 11 to separate the effects of matrix and enzyme concentration on specific activity. Depletion of the substrate was calculated from the detected concentration of 4-MU and the initial concentration of the substrate.

FIG. 13A depicts a representative data set collected from the experiment to separate the effects of matrix and enzyme concentration on specific activity where the enzyme concentration was varied and the buffer concentration was varied. FIG. 13B depicts a representative data set collected from the experiment to separate the effects of matrix and enzyme concentration on specific activity where the enzyme concentration was varied and the buffer concentration was constant. FIG. 13C depicts a representative data set collected from the experiment to separate the effects of matrix and enzyme concentration on specific activity where the enzyme concentration was constant and the buffer concentration was varied.

### DEFINITIONS

The patent and scientific literature referred to herein establishes knowledge that is available to those of skill in the art. The issued U.S. patents, allowed applications, published foreign applications, and references, including GenBank or other database sequences, that are cited herein are hereby incorporated by reference to the same extent as if each was specifically and individually indicated to be incorporated by reference. As used herein, the recitation of a numerical range for a variable is intended to convey that the invention can be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

As used herein, unless specifically indicated otherwise, the word “or” is used in the inclusive sense of “and/or” and not the exclusive sense of “either/or.”

As used herein, the term “approximately” or “about” means within ±10% of the value it modifies. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%.” Unless otherwise clear from the context, all numerical values provided herein are modified by the term “about”.

As used herein, the singular forms “a,” “an” and “the” include plural referents unless the context clearly dictates otherwise.

The terms “one or more”, “at least one”, “more than one”, and the like are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

As used herein, the phrase “biologically active” refers to a characteristic of any substance that has activity in a biological system (e.g., cell culture, organism, etc.). For instance, a substance that, when administered to an organism, has a biological effect on that organism, is considered to be biologically active. Biological activity can also be determined by in vitro assays (for example, in vitro enzymatic assays such as sulfate release assays). In particular embodiments, where a protein or polypeptide is biologically active, a portion of that protein or polypeptide that shares at least one biological activity of the protein or polypeptide is typically referred to as a “biologically active” portion. In some embodiments, a protein is produced and/or purified from a cell culture system, which displays biologically activity when administered to a subject. In some embodiments, a protein requires further processing in order to become biologically active. In some embodiments, a protein requires posttranslational modification such as, but is not limited to, glycosylation (e.g., sialyation), farnysylation, cleavage, folding, formylglycine conversion and combinations thereof, in order to become biologically active. In some embodiments, a protein produced as a proform (i.e. immature form), may require additional modification to become biologically active.

As used herein, the term “cation-independent mannose-6-phosphate receptor (CI-MPR)” refers to a cellular receptor that binds mannose-6-phosphate (M6P) tags on acid hydrolase precursors in the Golgi apparatus that are destined for transport to the lysosome. In addition to mannose-6-phosphates, the CI-MPR also binds other proteins including IGF-II. The CI-MPR is also known as “M6P/IGF-II receptor,” “CI-MPR/IGF-II receptor,” “IGF-II receptor” or “IGF2 Receptor.” These terms and abbreviations thereof are used interchangeably herein.

As used herein, the term “chromatography” refers to a technique for separation of mixtures. Typically, the mixture is dissolved in a fluid called the “mobile phase,” which carries it through a structure holding another material called the “stationary phase.” Column chromatography is a separation technique in which the stationary bed is within a tube, i.e., column.

As used herein, the term “diluent” refers to a pharmaceutically acceptable (e.g., safe and non-toxic for administration to a human) diluting substance useful for the preparation of a reconstituted formulation. Exemplary diluents include sterile water, bacteriostatic water for injection (BWFI), a pH buffered solution (e.g. phosphate-buffered saline), sterile saline solution, Ringer's solution or dextrose solution.

As used herein, the term “elution” refers to the process of extracting one material from another by washing with a solvent. For example, in ion-exchange chromatography, elution is a process to wash loaded resins to remove captured ions.

As used herein, the term “eluate” refers to a combination of mobile phase “carrier” and the analyte material that emerges from the chromatography, typically as a result of eluting.

As used herein, the term “enzyme replacement therapy (ERT)” refers to any therapeutic strategy that corrects an enzyme deficiency by providing the missing enzyme. Once administered, enzyme is taken up by cells and transported to the lysosome, where the enzyme acts to eliminate material that has accumulated in the lysosomes due to the enzyme deficiency. Typically, for lysosomal enzyme replacement therapy to be effective, the therapeutic enzyme is delivered to lysosomes in the appropriate cells in target tissues where the storage defect is manifest.

As used herein, the terms “equilibrate” or “equilibration” in relation to chromatography refer to the process of bringing a first liquid (e.g., buffer) into balance with another, generally to achieve a stable and equal distribution of components of the liquid (e.g., buffer). For example, in some embodiments, a chromatographic column may be equilibrated by passing one or more column volumes of a desired liquid (e.g., buffer) through the column.

As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of lysosomal storage disease as the individual being treated, who is about the same age as the individual being treated (to ensure that the stages of the disease in the treated individual and the control individual(s) are comparable).

As used herein, the term “impurities” refers to substances inside a confined amount of liquid, gas, or solid, which differ from the chemical composition of the target material or compound. Impurities are also referred to as contaminants.

As used herein, the term “linker” refers to, in a fusion protein, an amino acid sequence other than that appearing at a particular position in the natural protein and is generally designed to be flexible or to interpose a structure, such as an a-helix, between two protein moieties. A linker is also referred to as a spacer.

As used herein, the term “load” refers to, in chromatography, adding a sample-containing liquid or solid to a column. In some embodiments, particular components of the sample loaded onto the column are then captured as the loaded sample passes through the column. In some embodiments, particular components of the sample loaded onto the column are not captured by, or “flow through”, the column as the loaded sample passes through the column.

As used herein, a “polypeptide,” “peptide” and “protein,” generally speaking, are used interchangeably herein to refer to a string of at least two amino acids attached to one another by a peptide bond. That is, a description directed to a polypeptide applies equally to a description of a peptide and a description of a protein, and vice versa. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

As used herein, the term “pool” in relation to chromatography refers to combining one or more fractions of fluid that has passed through a column together. For example, in some embodiments, one or more fractions which contain a desired component of a sample that has been separated by chromatography (e.g., “peak fractions”) can be “pooled” together generate a single “pooled” fraction.

As used herein, the term “replacement enzyme” refers to any enzyme that can act to replace at least in part the deficient or missing enzyme in a disease to be treated. In some embodiments, the term “replacement enzyme” refers to any enzyme that can act to replace at least in part the deficient or missing lysosomal enzyme in a lysosomal storage disease to be treated. In some embodiments, a replacement enzyme is capable of reducing accumulated materials in mammalian lysosomes or that can rescue or ameliorate one or more lysosomal storage disease symptoms. Replacement enzymes suitable for the invention include both wild-type or modified lysosomal enzymes and can be produced using recombinant and synthetic methods or purified from nature sources. A replacement enzyme can be a recombinant, synthetic, gene-activated or natural enzyme.

As used herein, the term “soluble” refers to the ability of a therapeutic agent to form a homogenous solution. In some embodiments, the solubility of the therapeutic agent in the solution into which it is administered and by which it is transported to the target site of action is sufficient to permit the delivery of a therapeutically effective amount of the therapeutic agent to the targeted site of action. Several factors can impact the solubility of the therapeutic agents. For example, relevant factors which may impact protein solubility include ionic strength, amino acid sequence and the presence of other co-solubilizing agents or salts (e.g., calcium salts). In some embodiments, therapeutic agents in accordance with the present invention are soluble in its corresponding pharmaceutical composition.

As used herein, the term “stable” refers to the ability of the therapeutic agent (e.g., a recombinant enzyme) to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In the context of a formulation a stable formulation is one in which the therapeutic agent therein essentially retains its physical and/or chemical integrity and biological activity upon storage and during processes (such as freeze/thaw, mechanical mixing and lyophilization). For protein stability, it can be measure by formation of high molecular weight (HMW) aggregates, loss of enzyme activity, generation of peptide fragments, and shift of charge profiles.

As used herein, the term “viral processing” refers to “viral removal,” in which viruses are simply removed from the sample, or “viral inactivation,” in which the viruses remain in a sample but in a non-infective form. In some embodiments, viral removal may utilize nanofiltration and/or chromatographic techniques, among others. In some embodiments, viral inactivation may utilize solvent inactivation, detergent inactivation, pasteurization, acidic pH inactivation, and/or ultraviolet inactivation, among others.

Unless otherwise defined, all technical and scientific terms used herein have the same meaning as commonly understood by one of ordinary skill in the art to which this application belongs and as commonly used in the art to which this application belongs; such art is incorporated by reference in its entirety. In the case of conflict, the present Specification, including definitions, will control.

### DETAILED DESCRIPTION

The present invention provides, among other things, improved methods of producing and purifying compositions for treating Hunter syndrome via enzyme replacement therapy. The present invention provides highly potent iduronate-2-sulfatase fusion protein (HIRMab-I2S fusion protein) that can effectively cross the blood brain barrier (BBB) for treatment of CNS symptoms associated with Hunter syndrome. In addition, the present invention provides a method of purifying sulfatase fusion protein (e.g. I2S-immunoglobulin fusion protein) from an impure preparation using a process based on one or more of affinity chromatography, cation-exchange chromatography, and multimodal chromatography. In some embodiments, the present invention provides a method of purifying I2S immunoglobulin fusion protein from an impure preparation by conducting Protein A chromatography, Capto SP ImpRes chromatography, and Capto Adhere chromatography. In some embodiments, the present invention provides processes that are capable of producing such highly potent iduronate-2-sulfatase fusion protein at a commercially viable scale. The present invention further provides purified I2S-immunoglobulin fusion protein and method of use.

Various aspects of the invention are described in further detail in the following subsections. The use of subsections is not meant to limit the invention. Each subsection may apply to any aspect of the invention. In this application, the use of “or” means “and/or” unless stated otherwise.

Iduronate-2-sulfatase (I2S)

A suitable I2S for the present invention is any protein or a portion of a protein that can substitute for at least partial activity of naturally-occurring Iduronate-2-sulfatase (I2S) protein or rescue one or more phenotypes or symptoms associated with I2S-deficiency. As used herein, the terms “an I2S enzyme” and “an I2S protein”, and grammatical equivalents, are used inter-changeably.

Typically, the human I2S protein is produced as a precursor form. The precursor form of human I2S contains a signal peptide (amino acid residues 1-25 of the full length precursor), a pro-peptide (amino acid residues 26-33 of the full length precursor), and a chain (residues 34-550 of the full length precursor) that may be further processed into the 42 kDa chain (residues 34-455 of the full length precursor) and the 14 kDa chain (residues 446-550 of the full length precursor). Typically, the precursor form is also referred to as full-length precursor or full-length I2S protein, which contains 550 amino acids. The amino acid sequences of the mature form (SEQ ID NO:1) having the signal peptide removed and full-length precursor (SEQ ID NO:2) of a typical wild-type or naturally-occurring human I2S protein are shown in Table 1. The signal peptide is underlined. In addition, the amino acid sequences of human I2S protein isoform a and b precursor are also provided in Table 1, SEQ ID NO:3 and 4, respectively.

TABLE 1
Human Iduronate-2-sulfatase

| | |
|---|---|
| Mature Form | SETQANSTTDALNVLLIIVDDLRPSLGCYGDKLVRSPNIDQL |
| | ASHSLLFQNAFAQQAVCAPSRVSFLTGRRPDTTRLYDFNSY |
| | WRVHAGNFSTIPQYFKENGYVTMSVGKVFHPGISSNHTDD |
| | SPYSWSFPPYHPSSEKYENTKTCRGPDGELHANLLCPVDVL |
| | DVPEGTLPDKQSTEQAIQLLEKMKTSASPFFLAVGYHKPHI |
| | PFRYPKEFQKLYPLENITLAPDPEVPDGLPPVAYNPWMDIR |
| | QREDVQALNISVPYGPIPVDFQRKIRQSYFASVSYLDTQVG |
| | RLLSALDDLQLANSTIIAFTSDHGWALGEHGEWAKYSNFD |
| | VATHVPLIFYVPGRTASLPEAGEKLFPYLDPFDSASQLMEP |
| | GRQSMDLVELVSLFPTLAGLAGLQVPPRCPVPSFHVELCRE |
| | GKNLLKHFRFRDLEEDPYLPGNPRELIAYSQYPRPSDIPQW |
| | NSDKPSLKDIKIMGYSIRTIDYRYTVWVGFNPDEFLANFSDI |
| | HAGELYFVDSDPLQDHNMYNDSQGGDLFQLLMP (SEQ ID |
| | NO: 1) |
| Full-Length | MPPPRTGRGLLWLGLVLSSVCVALGSETQANSTTDALNVL |
| Precursor | LIIVDDLRPSLGCYGDKLVRSPNIDQLASHSLLFQNAFAQQ |
| | AVCAPSRVSFLTGRRPDTTRLYDFNSYWRVHAGNFSTIPQY |
| | FKENGYVTMSVGKVFHPGISSNHTDDSPYSWSFPPYHPSSE |
| | KYENTKTCRGPDGELHANLLCPVDVLDVPEGTLPDKQSTE |
| | QAIQLLEKMKTSASPFFLAVGYHKPHIPFRYPKEFQKLYPL |
| | ENITLAPDPEVPDGLPPVAYNPWMDIRQREDVQALNISVPY |
| | GPIPVDFQRKIRQSYFASVSYLDTQVGRLLSALDDLQLANS |
| | TIIAFTSDHGWALGEHGEWAKYSNFDVATHVPLIFYVPGRT |
| | ASLPEAGEKLFPYLDPFDSASQLMEPGRQSMDLVELVSLFP |
| | TLAGLAGLQVPPRCPVPSFHVELCREGKNLLKHFRFRDLEE |
| | DPYLPGNPRELIAYSQYPRPSDIPQWNSDKPSLKDIKIMGYS |
| | IRTIDYRYTVWVGFNPDEFLANFSDIHAGELYFVDSDPLQD |
| | HNMYNDSQGGDLFQLLMP (SEQ ID NO: 2) |
| Isoform a | MPPPRTGRGLLWLGLVLSSVCVALGSETQANSTTDALNVL |
| Precursor | LIIVDDLRPSLGCYGDKLVRSPNIDQLASHSLLFQNAFAQQ |
| | AVCAPSRVSFLTGRRPDTTRLYDFNSYWRVHAGNFSTIPQY |
| | FKENGYVTMSVGKVFHPGISSNHTDDSPYSWSFPPYHPSSE |
| | KYENTKTCRGPDGELHANLLCPVDVLDVPEGTLPDKQSTE |
| | QAIQLLEKMKTSASPFFLAVGYHKPHIPFRYPKEFQKLYPL |
| | ENITLAPDPEVPDGLPPVAYNPWMDIRQREDVQALNISVPY |
| | GPIPVDFQEDQSSTGFRLKTSSTRKYK (SEQ ID NO: 3) |
| Isoform b | MPPPRTGRGLLWLGLVLSSVCVALGSETQANSTTDALNVL |
| Precursor | LIIVDDLRPSLGCYGDKLVRSPNIDQLASHSLLFQNAFAQQ |
| | AVCAPSRVSFLTGRRPDTTRLYDFNSYWRVHAGNFSTIPQY |
| | FKENGYVTMSVGKVFHPGISSNHTDDSPYSWSFPPYHPSSE |
| | KYENTKTCRGPDGELHANLLCPVDVLDVPEGTLPDKQSTE |
| | QAIQLLEKMKTSASPFFLAVGYHKPHIPFRYPKEFQKLYPL |
| | ENITLAPDPEVPDGLPPVAYNPWMDIRQREDVQALNISVPY |
| | GPIPVDFQRKIRQSYFASVSYLDTQVGRLLSALDDLQLANS |
| | TIIAFTSDHGFLMRTNT (SEQ ID No: 4) |

In some embodiments, a suitable I2S for the present invention is mature human I2S protein (SEQ ID NO:1). As disclosed herein, SEQ ID NO:1 represents the canonical amino acid sequence for the human I2S protein. In some embodiments, the I2S protein may be a splice isoform and/or variant of SEQ ID NO:1, resulting from transcription at an alternative start site within the 5′ UTR of the I2S gene. In some embodiments, an I2S protein may be a homologue or an analogue of mature human I2S protein. For example, a homologue or an analogue of mature human I2S protein may be a modified mature human I2S protein containing one or more amino acid substitutions, deletions, and/or insertions as compared to a wild-type or naturally-occurring I2S protein (e.g., SEQ ID NO:1), while retaining substantial I2S protein activity. In some embodiments, an I2S protein is substantially homologous to mature human I2S protein (SEQ ID NO:1). In some embodiments, an I2S protein has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:1. In some embodiments, an I2S protein is substantially identical to mature human I2S protein (SEQ ID NO:1). In some embodiments, an I2S protein has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:1. In some embodiments, an I2S protein contains a fragment or a portion of mature human I2S protein.

Alternatively, a suitable I2S is full-length I2S protein. In some embodiments, a suitable I2S may be a homologue or an analogue of full-length human I2S protein. For example, a homologue or an analogue of full-length human I2S protein may be a modified full-length human I2S protein containing one or more amino acid substitutions, deletions, and/or insertions as compared to a wild-type or naturally-occurring full-length I2S protein (e.g., SEQ ID NO:2), while retaining substantial I2S protein activity. Thus, in some embodiments, an I2S protein is substantially homologous to full-length human I2S protein (SEQ ID NO:2). For example, an I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:2. In some embodiments, an 12S protein is substantially identical to SEQ ID NO:2. For example, an 12S protein may have an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:2. In some embodiments, an I2S protein contains a fragment or a portion of full-length human I2S protein. As used herein, a full-length I2S protein typically contains signal peptide sequence. In some embodiments, a suitable I2S is human I2S isoform a protein. In some embodiments, a suitable I2S may be a homologue or an analogue of human I2S isoform a protein. For example, a homologue or an analogue of human I2S isoform a protein may be a modified human I2S isoform a protein containing one or more amino acid substitutions, deletions, and/or insertions as compared to a wild-type or naturally-occurring human I2S isoform a protein (e.g., SEQ ID NO:3), while retaining substantial I2S protein activity. Thus, in some embodiments, a suitable I2S is substantially homologous to human I2S isoform a protein (SEQ ID NO:3). For example, a suitable I2S may have an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:3. In some embodiments, a suitable I2S is substantially identical to SEQ ID NO:3. For example, a suitable I2S may have an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:3. In some embodiments, a suitable I2S contains a fragment or a portion of human I2S isoform a protein. As used herein, a human I2S isoform a protein typically contains a signal peptide sequence.

In some embodiments, a suitable I2S is human I2S isoform b protein. In some embodiments, a suitable I2S may be a homologue or an analogue of human I2S isoform b protein. For example, a homologue or an analogue of human I2S isoform b protein may be a modified human I2S isoform b protein containing one or more amino acid substitutions, deletions, and/or insertions as compared to a wild-type or naturally-occurring human I2S isoform b protein (e.g., SEQ ID NO:4), while retaining substantial I2S protein activity. In some embodiments, an I2S protein is substantially homologous to human I2S isoform b protein (SEQ ID NO:4). For example, an I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:4. In some embodiments, an I2S protein is substantially identical to SEQ ID NO:4. For example, an I2S protein may have an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:4. In some embodiments, an 12S protein contains a fragment or a portion of human 12S isoform b protein. As used herein, a human 12S isoform b protein typically contains a signal peptide sequence.

The skilled artisan will realize that conservative amino acid substitutions may be made in I2S polypeptides to provide functionally equivalent variants of the foregoing polypeptides, i.e., the variants retain the functional capabilities of the I2S polypeptides. As used herein, a conservative amino acid substitution refers to an amino acid substitution which does not significantly alter the tertiary structure and/or activity of the polypeptide. Variants can be prepared according to methods for altering polypeptide sequence known to one of ordinary skill in the art, and include those that are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the I2S polypeptides include conservative amino acid substitutions of SEQ ID NO:2. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

### I2S-Immunoglobulin Fusion Protein

The present invention may be used to produce any purified sulfatase-immunoglobulin fusion protein (e.g. I2S-immunoglobulin fusion protein). In particular, the present invention may be used to produce a fusion protein in which I2S is fused to an immunoglobulin that is capable of crossing the blood brain barrier (BBB), with or without intervening sequence. As used herein, the “blood-brain barrier” or “BBB” refers to the barrier between the peripheral circulation and the brain and spinal cord which is formed by tight junctions within the brain capillary endothelial cell plasma membranes and creates an extremely tight barrier that restricts the transport of molecules into the brain; the BBB is so tight that it is capable of restricting even molecules as small as urea, molecular weight of 60 Da. The blood-brain barrier within the brain, the blood spinal cord barrier within the spinal cord, and the blood-retinal barrier within the retina, are contiguous capillary barriers within the central nervous system (CNS), and are collectively referred to as the blood-brain barrier or BBB.

### Immunoglobulin

The BBB has been shown to have specific receptors that allow the transport of macromolecules from the blood to the brain. For example, any immunoglobulin that may trigger receptor-mediated endocytosis and transcytosis can be used. Exemplary endogenous BBB receptor-mediated transport systems useful in the invention include those that transport insulin, transferrin, insulin-like growth factors 1 and 2 (IGF1 and IGF2), leptin, and lipoproteins. Thus, in some embodiments, a suitable immunoglobulin according to the present invention binds to an endogenous BBB receptor, thereby crossing the BBB. Various endogenous BBB receptors are known in the art and are well characterized. For example, Insulin receptors and their extracellular, insulin binding domain (ECD) have been extensively characterized in the art both structurally and functionally. See, e.g., Yip et al (2003), J. Biol. Chem, 278(30):27329-27332; and Whittaker et al. (2005), J. Biol. Chem, 280(22):20932-20936. The amino acid and nucleotide sequences of the human insulin receptor can be found under GenBank accession No. NM 000208.

As non-limiting examples, a suitable immunoglobulin according to the present invention binds to an insulin receptor, a transferrin receptor, an insulin-like growth factors 1 and 2 (IGF1 and IGF2) receptor, a leptin receptor, and/or a lipoproteins receptor. In other embodiments, a suitable immunoglobulin may be a single domain antibody (sdAb), such as FC5 or FC44.

As used herein, the term “immunoglobulin” refers to an antibody, or a portion of an antibody. An “antibody” is a polypeptide that includes canonical immunoglobulin sequence elements sufficient to confer specific binding to a particular target antigen. Antibodies include two heavy chain polypeptides and two light chain polypeptides (about 25 kD each) that associate with each other into what is commonly referred to as a “Y-shaped” structure. Each heavy chain is comprised of at least four domains—a variable (VH) domain and three constant domains: CH1, CH2, and CH3. Each light chain is comprised of two domains—a variable (VL) domain and a constant (CL) domain. Each variable domain (whether on the heavy or light chain) contains three hypervariable loops known as “complement determining regions” (CDR1, CDR2, and CDR3) and four somewhat invariant “framework” regions (FR1, FR2, FR3, and FR4). The fragment crystallizable (Fc) region includes the CH2 and CH3 domains of two heavy chains. The Fc region of naturally-occurring antibodies binds to elements of the complement system, and also to receptors on effector cells, including for example effector cells that mediate cytotoxicity. An “immunoglobulin” can, therefore, also refer to a structural unit (e.g., a heavy or light chain), a fragment (e.g., a Fc, Fab, F(ab′)2, F(ab)2, or Fab′), or a region (e.g., a variable region, more specifically, a CDR) of an antibody, or recombinant antibodies including, but not limited to, scFvs, scFv-Fc fusions, diabodies, triabodies and tetrabodies. An “antibody” can also be a bispecific antibody, which is an artificial protein that is composed of fragments of two different antibodies and consequently bind to two different types of antigens. An antibody can be different types referred to as isotypes or classes. There are five antibody isotypes known as IgA, IgD, IgE, IgG, and IgM in placental mammals. Valency is the number of antigen binding cites of the antibody. There could be different isotypes that therefore contain multiple antigen binding cites. For example, IgM is a pentamer of five “Y” shaped monomers; therefore, the complete IgM protein contains 10 heavy chains, 10 light chains and 10 antigen binding arms giving IgM a valency of 10.

It will be apparent to one of ordinary skill in the art, that the present invention also encompasses F(ab′)2, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies. In some embodiments, the antibody of the present invention includes only one CDR.

In some embodiments, an antibody of the present invention is a monoclonal antibody (Mab), typically a chimeric human-mouse antibody derived by humanization of a mouse monoclonal antibody. Such antibodies are obtained from, e.g., transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas.

For use in humans, a chimeric antibody (e.g., HIR Ab, other antibodies capable of crossing the BBB) is preferred that contains enough human sequence that it is not significantly immunogenic when administered to humans, e.g., about 80% human and about 20% mouse, or about 85% human and about 15% mouse, or about 90% human and about 10% mouse, or about 95% human and 5% mouse, or greater than about 95% human and less than about 5% mouse. A more highly humanized form of the antibody (e.g., HIR Ab, other antibodies capable of crossing the BBB) can also be engineered, and the humanized antibody (e.g. HIR Ab) has activity comparable to the murine HIR Ab and can be used in embodiments of the invention. See, e.g., U.S. Patent Application Publication Nos. 2004-0101904, filed Nov. 27, 2002 and 2005-0142141, filed Feb. 17, 2005. Humanized antibodies to the human BBB insulin receptor with sufficient human sequences for use in the invention are described in, e.g., Boado et al. (2007), Biotechnol Bioeng, 96(2):381-391.

### HIRMab-I2S-Fusion Protein

In embodiments, the antibody of the current disclosure is a human insulin receptor monoclonal antibody fused with I2S (HIRMab-I2S). HIRMab-I2S is defined by an amino acid sequence that is about 50%, 55%, 60%, 65%, 70%, 75%, 80%, 85%, 90%, 91%, 92%, 93%, 94%, 95%, 96%, 97%, 98%, 99%, or 100% identical SEQ ID NO: 5. In embodiments, the HIRMab-I2S sequence is identical to SEQ ID NO: 5. SEQ ID NO: 5 encodes an IgG HC fusion protein (970 amino acids), wherein the mature 525 amino acid human iduronate-2-sulfatase (I2S) enzyme is fused to the carboxy terminus of the heavy chain (HC) of a chimeric human insulin receptor monoclonal antibody. The amino acid sequence of the HIRMab-I2S is shown in Table 2 below.

In embodiments, the antibody of the current disclosure includes an amino acid sequence of a recombinant human IgG light chain. The recombinant human IgG light chain is defined by an amino acid sequence that is about 50%, 55%, 60%, 65%, 70%, 75%, 80%, 85%, 90%, 91%, 92%, 93%, 94%, 95%, 96%, 97%, 98%, 99%, or 100% identical SEQ ID NO: 6. In embodiments, the recombinant human IgG light chain is identical to SEQ ID NO: 6.

TABLE 2
Human Insulin Receptor Monoclonal Antibody
Fused with I2S (HIRMab-I2S)

HIRMab-I2S Amino Acid Sequence*
MDWTWRVFCLLAVAPGAHSQVQLQQSGPELVKPGALVKISCKASGYTFTNYDIH
WVKQRPGQGLEWIGWIYPGDGSTKYNEKFKGKATLTADKSSSTAYMHLSSLTSE
KSAVYFCAREWAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGN
VFSCSVMHEALHNHYTQKSLSLSPGSSSSETQANSTTDALNVLLIIVDDLRPSLGC
YGDKLVRSPNIDQLASHSLLFQNAFAQQAVCAPSRVSFLTGRRPDTTRLYDFNSY
WRVHAGNFSTIPQYFKENGYVTMSVGKVFHPGISSNHTDDSPYSWSFPPYHPSSE
KYENTKTCRGPDGELHANLLCPVDVLDVPEGTLPDKQSTEQAIQLLEKMKTSASP
FFLAVGYHKPHIPFRYPKEFQKLYPLENITLAPDPEVPDGLPPVAYNPWMDIRQRE
DVQALNISVPYGPIPVDFQRKIRQSYFASVSYLDTQVGRLLSALDDLQLANSTIIAF
TSDHGWALGEHGEWAKYSNFDVATHVPLIFYVPGRTASLPEAGEKLFPYLDPFDS
ASQLMEPGRQSMDLVELVSLFPTLAGLAGLQVPPRCPVPSFHVELCREGKNLLKH
FRFRDLEEDPYLPGNPRELIAYSQYPRPSDIPQWNSDKPSLKDIKIMGYSIRTIDYR
YTVWVGFNPDEFLANFSDIHAGELYFVDSDPLQDHNMYNDSQGGDLFQLLMP
(SEQ ID NO: 11)
Recombinant Human IgG Light Chain
METPAQLLFLLLLWLPDTTGDIQMTQSPSSLSASLGERVSLTCRASQDIGGNLYW
LQQGPDGTIKRLIYATSSLDSGVPKRFSGSRSGSDYSLTISSLESEDFVDYYCLQYS
SSPWTFGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSS
PVTKSFNRGEC
(SEQ ID NO: 12)
*The underlined portion of the SEQ ID NO: 5 corresponds to the mature 525 amino acid sequence of human iduronate-2-sulfatase (I2S).

The skilled artisan will realize that conservative amino acid substitutions may be made in any of the polypeptides presented herein (e.g. HIRMab-I2S, Recombinant Human IgG Light Chain, and/or I2S) to provide functionally equivalent variants of the foregoing polypeptides, i.e, the variants retain the functional capabilities.

In embodiments, the HIR antibodies or HIRMab-I2S fusion protein contains both a heavy chain and a light chain corresponding to any of the above-mentioned HIR heavy chains and HIR light chains.

In some embodiments, the immunoglobulin comprises a chimeric monoclonal antibody that binds to the Human Insulin Receptor (HIR). The HIR can mediate transport across the Blood Brain Barrier via endogenous brain capillary endothelial insulin receptors. The HIR can mediate transport via endogenous neuronal insulin receptors.

HIR antibodies used in the invention may be glycosylated or non-glycosylated. If the antibody is glycosylated, any pattern of glycosylation that does not significantly affect the function of the antibody may be used. Glycosylation can occur in the pattern typical of the cell in which the antibody is made, and may vary from cell type to cell type. For example, the glycosylation pattern of a monoclonal antibody produced by a mouse myeloma cell can be different than the glycosylation pattern of a monoclonal antibody produced by a transfected Chinese hamster ovary (CHO) cell. In some embodiments, the antibody is glycosylated in the pattern produced by a transfected Chinese hamster ovary (CHO) cell.

One of ordinary skill in the art will appreciate that current technologies permit a vast number of sequence variants of candidate antibodies (e.g., HIR Ab, other antibodies capable of crossing the BBB) can be generated be (e.g., in vitro) and screened for binding to a target antigen such as the ECD of the human insulin receptor or an isolated epitope thereof. See, e.g., Fukuda et al. (2006) “In vitro evolution of single-chain antibodies using mRNA display,” Nuc. Acid Res., 34(19) (published online) for an example of ultra-high throughput screening of antibody sequence variants. See also, Chen et al. (1999), “In vitro scanning saturation mutagenesis of all the specificity determining residues in an antibody binding site,” Prot Eng, 12(4): 349-356. An insulin receptor ECD can be purified as described in, e.g., Coloma et al. (2000) Pharm Res, 17:266-274, and used to screen for HIR Abs and HIR Ab sequence variants of known HIR Abs.

Accordingly, in some embodiments, a genetically engineered HIR Ab, with the desired level of human sequences, is fused to an I2S, to produce a recombinant fusion antibody that is a bi-functional molecule. The HIR Ab-I2S fusion antibody: (i) binds to an extracellular domain of the human insulin receptor; (ii) catalyzes hydrolysis of linkages in dermatan and/or heparan sulfate; and (iii) is able to cross the BBB, via transport on the BBB HIR, and retain I2S activity once inside the brain, following peripheral administration.

### Linker

An I2S fusion protein (e.g., HIRMAb-I2S) described herein can include a covalent linkage between immunoglobulin and I2S. A covalent linkage may be to the carboxy or amino terminal of the immunoglobulin (e.g., HIR antibody) and the amino or carboxy terminal of I2S and the linkage allows the immunoglobulin to bind to the ECD of a receptor and cross the blood brain barrier, and allows the I2S to retain a therapeutically useful portion of its activity. In certain embodiments, the covalent link is between a heavy chain of the antibody and the I2S. In some embodiments, the covalent link is between a light chain of an antibody and the I2S. Any suitable linkage may be used, e.g., carboxy terminus of light chain to amino terminus of I2S, carboxy terminus of heavy chain to amino terminus of I2S, amino terminus of light chain to amino terminus of I2S, amino terminus of heavy chain to amino terminus of I2S, carboxy terminus of light chain to carboxy terminus of I2S, carboxy terminus of heavy chain to carboxy terminus of I2S, amino terminus of light chain to carboxy terminus of I2S, or amino terminus of heavy chain to carboxy terminus of I2S. In some embodiments, the linkage is from the carboxy terminus of the HC to the amino terminus of the I2S.

In some embodiments, a fusion protein described herein includes a linker or spacer between I2S and immunoglobulin as part of the fused amino acid sequence. In some embodiments, a fusion protein described herein does not include a linker or a spacer between the fused proteins. Typically, a suitable linker or spacer is an amino acid linker or spacer (also referred to as a peptide linker or spacer). An amino acid (or peptide) linker or spacer is generally designed to be flexible or to interpose a structure, such as an alpha-helix, between the two protein moieties. A suitable peptide sequence linker may be at least 0, 1, 2, 3, 4, 5, 6, 7, 8, 9, 10, 15, 20, 25, 30, 35, 40, 45, 50 or more amino acids in length. In some embodiments, a peptide linker is less than 50, 45, 40, 35, 30, 35, 20, 15, 14, 13, 12, 11, 10, 9, 8, 7, 6, 5, 4, 3, 2, or 1 amino acids in length. In some embodiments, the I2S is directly linked to the targeting antibody, and is therefore 0 amino acids in length. In some embodiments, a suitable peptide linker may be, for example, 10-50 (e.g., 10-20, 10-25, 10-30, 10-35, 10-40, 10-45, 10-50) amino acids in length.

In some embodiments, a suitable linker comprises glycine, serine, and/or alanine residues in any combination or order. In some cases, the combined percentage of glycine, serine, and alanine residues in the linker is at least 5%, 10%, 15%, 20%, 25%, 30%, 40%, 50%, 60%, 70%, 75%, 80%, 90%, or 95% of the total number of residues in the linker. In some embodiments, the combined percentage of glycine, serine, and alanine residues in the linker is at least 50%, 60%, 70%, 75%, 80%, 90%, or 95% of the total number of residues in the linker. In some embodiments, any number of combinations of amino acids (including natural or synthetic amino acids) can be used for the linker. In some embodiments, a two amino acid linker is used. In some embodiments, a linker has the sequence Ser-Ser. In some embodiments, a two amino acid linker comprises glycine, serine, and/or alanine residues in any combination or order (e.g., Gly-Gly, Ser-Gly, Gly-Ser, Ser-Ser, Ala-Ala, Ser-Ala, or Ala-Ser linker). In some embodiments, a two amino acid linker consists of one glycine, serine, and/or alanine residue along with another amino acid (e.g., Ser-X, where X is any known amino acid). In still other embodiments, the two-amino acid linker consists of any two amino acids (e.g., X-X), except Gly, Ser, or Ala.

As described herein, in some embodiments, a linker is greater than two amino acids in length. Such linker may also comprise glycine, serine, and/or alanine residues in any combination or order, as described further herein. In some embodiments, a linker includes one glycine, serine, and/or alanine residue along with other amino acids (e.g., Ser-nX, where X is any known amino acid, and n is the number of amino acids). In other embodiments, a linker consists of any two amino acids (e.g., X-X). In some embodiments, said any two amino acids are Gly, Ser, or Ala, in any combination or order, and within a variable number of amino acids intervening between them. In some embodiments, a suitable linker includes at least one Gly, at least one Ser, and/or at least one Ala. In some embodiments, a linker includes at least 1, 2, 3, 4, 5, 6, 7, 8, 9, or 10 Gly, Ser, and/or Ala residues. In some embodiments, a suitable linker comprises Gly and Ser in repeating sequences, in any combination or number, such as (Gly4Ser)3, or other variations.

In some embodiments, a linker or spacer can include the sequence GGGGGAAAAGGGG (SEQ ID NO:7), GAP (SEQ ID NO:8), or GGGGGP (SEQ ID NO:9). In some embodiments, various short linker sequences can be present in tandem repeats. For example, a suitable linker may contain the amino acid sequence of GGGGGAAAAGGGG (SEQ ID NO:7) present in tandem repeats. In some embodiments, a suitable linker may further contain one or more GAP sequences that frame the sequence of GGGGGAAAAGGGG (SEQ ID NO:7). For example, a suitable linker may contain amino acid sequence of

(SEQ ID NO: 10)
GAPGGGGGAAAAGGGGGAPGGGGGAAAAGGGGGAPGGGGGAAAAGGGGG
AP.

In some embodiments, a suitable linker or spacer may contain a sequence at least 50%, 55%, 60%, 65%, 70%, 75%, 80%, 85%, 90%, 95%, 98%, or 99% identical to any of the linker sequences described herein.

Additional exemplary linker or spacer sequences are described in U.S. Pat. No. 8,580,922, which is incorporated herein by reference.

A linker for use in the present invention may also be designed by using any method known in the art. For example, there are multiple publicly-available programs for determining optimal amino acid linkers in the engineering of fusion proteins. Publicly-available computer programs (such as the LINKER program) that automatically generate the amino acid sequence of optimal linkers based on the user's input of the sequence of the protein and the desired length of the linker may be used for the present methods and compositions. Often, such programs may use observed trends of naturally-occurring linkers joining protein subdomains to predict optimal protein linkers for use in protein engineering. In some cases, such programs use other methods of predicting optimal linkers. Examples of some programs suitable for predicting a linker for the present invention are described in the art, see, e.g., Xue et al. (2004) Nucleic Acids Res. 32, W562-W565 (Web Server issue providing internet link to LINKER program to assist the design of linker sequences for constructing functional fusion proteins); George and Heringa, (2003), Protein Engineering, 15(11):871-879 (providing an internet link to a linker program and describing the rational design of protein linkers); Argos, (1990), J. Mol. Biol. 211:943-958; Arai et al. (2001) Protein Engineering, 14(8):529-532; Crasto and Feng, (2000) Protein Engineering 13(5):309-312.

A peptide linker sequence may include a protease cleavage site; however, this is not a requirement for maintaining I2S activity.

### Lysosomal Targeting Moiety

In some embodiments, a suitable fusion protein of the present invention further includes a lysosomal targeting moiety. Typically, a lysosomal targeting moiety refers to a moiety that binds to a receptor on the surface of target cells to facilitate cellular uptake and/or lysosomal targeting. For example, such a receptor may be the cation-independent mannose-6-phosphate receptor (CI-MPR) which binds the mannose-6-phosphate (M6P) residues. In addition, the CI-MPR also binds other proteins including IGF-II. Thus, in some embodiments, an I2S fusion protein described herein contains M6P residues on the surface of the protein. In particular, a fusion protein described herein may contain bis-phosphorylated oligosaccharides which have higher binding affinity to the CI-MPR. In some embodiments, a lysosomal targeting moiety is any protein, peptide, or fragment thereof that binds the CI-M6PR, in a mannose-6-phosphate-dependent manner. In some embodiments, a lysosomal targeting moiety is any protein, peptide, or fragment thereof that binds directly to a region, domain and/or extracellular portion of CI-M6PR. In some embodiments, a lysosomal targeting moiety is any protein, peptide, or fragment thereof that binds directly to a region, domain and/or extracellular portion of CI-M6PR via a M6P residue. In some embodiments, the M6P residue is a 2-mannose-6-phosphate residue.

In some embodiments, a lysosomal targeting moiety is any protein, peptide, or fragment thereof that binds the CI-M6PR, in a mannose-6-phosphate-independent manner. Suitable lysosomal targeting moieties may be derived from proteins or peptides including, but not limited to, IGF-II, IGF-I, ApoE, TAT, RAP, p97, Plasminogen, Leukemia Inhibitory Factor Peptide (LIF), Cellular Repressor of E1A-Stimulated Genes Peptide (CREG), Human Sortlin-1 Propeptide (SPP), Human Prosaposin peptide (SapDC) and Progranulin.

Various additional lysosomal targeting moieties are known in the art and can be used to practice the present invention. For example, certain peptide-based lysosomal targeting moieties are described in U.S. Pat. Nos. 7,396,811, 7,560,424, and 7,629,309; U.S. Application Publication Nos. 2003-0082176, 2004-0006008, 2003-0072761, 20040005309, 2005-0281805, 2005-0244400, and international publications WO 03/032913, WO 03/032727, WO 02/087510, WO 03/102583, WO 2005/078077, WO/2009/137721, the entire disclosures of which are incorporated herein by reference.

In some embodiments, a lysosomal targeting moiety is any peptide that is M6P phosphorylated by the cell. In some embodiments, the peptide is capable of binding to the CI-M6PR. In some embodiments, the peptide is an amino acid sequence found within a protein selected from the group consisting of Cathepsin B, Cathepsin D, Cathepsin L, Beta-Glucuroidase, Beta-Mannosidase, Alpha-Fucosidase, Beta-Hexosaminidase, Arylsulfatase, Beta-Galactosidase, Phosphomannan, Latent TGFbeta, Leukemia Inhibitory Factor, Proliferin, Prorenin, Herpes Simplex Virus, PI-LLC cleaved GPI anchor, Retinoic Acid, IGFII, Plasminogen, Thyroglobulin, TGFbetaR-V, CD87, GTP-binding Proteins (Gi-1, Gi-2 and Gi-3), HA-I Adaptin, HA-II Adaptin and combinations thereof. In some embodiments, the amino acid sequence includes a domain, fragment, region or segment of one or more proteins selected from the group consisting of Cathepsin B, Cathepsin D, Cathepsin L, Beta-Glucuroidase, Beta-Mannosidase, Alpha-Fucosidase, Beta-Hexosaminidase, Arylsulfatase, Beta-Galactosidase, Phosphomannan, Latent TGFbeta, Leukemia Inhibitory Factor, Proliferin, Prorenin, Herpes Simplex Virus, PI-LLC cleaved GPI anchor, Retinoic Acid, IGFII, Plasminogen, Thyroglobulin, TGFbetaR-V, CD87, GTP-binding Proteins (Gi-1, Gi-2 and Gi-3), HA-I Adaptin, HA-II Adaptin and combinations thereof. In some embodiments, the polypeptide is produced synthetically. In some embodiments, the polypeptide is produced recombinantly. Both approaches are widely used in the art and described, for example, in Sambrook et al., Molecular Cloning: A Laboratory Manual, Cold Spring Harbor Laboratory Press (2001).

In some embodiments, a suitable lysosomal targeting moiety may provide additional glycosylation sites to facilitate binding to the M6P receptor. Any peptide may be used within the scope of the present invention as long as it has a N-linked glycosylation site. N-linked glycosylation sites may be predicted by computer algorithms and software, many of which are generally known in the art. Alternatively, N-linked glycosylation may be determined experimentally using any one of the many assays generally known in the art.

### Production of I2S Immunoglobulin Fusion Proteins

The present invention may be used to purify I2S immunoglobulin fusion protein produced by various means. For example, an I2S immunoglobulin fusion protein may be produced by utilizing a host cell system engineered to express an I2S immunoglobulin fusion protein. As used herein, the term “host cells” refers to cells that can be used to produce I2S immunoglobulin fusion protein described herein. In particular, host cells are suitable for producing I2S immunoglobulin fusion protein described herein at a large scale. Suitable host cells can be derived from a variety of organisms, including, but not limited to, mammals, plants, birds (e.g., avian systems), insects, yeast, and bacteria. In some embodiments, host cells are mammalian cells.

### Mammalian Cell Lines

Any mammalian cell or cell type susceptible to cell culture, and to expression of polypeptides, may be utilized in accordance with the present invention as a host cell. Non-limiting examples of mammalian cells that may be used in accordance with the present invention include human embryonic kidney 293 cells (HEK293), HeLa cells; BALB/c mouse myeloma line (NSW, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells (CHO); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). In some embodiments, a suitable mammalian cell is not a endosomal acidification-deficient cell.

Additionally, any number of commercially and non-commercially available hybridoma cell lines that express polypeptides or proteins may be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

### Non-Mammalian Cell Lines

Any non-mammalian derived cell or cell type susceptible to cell culture, and to expression of polypeptides, may be utilized in accordance with the present invention as a host cell. Non-limiting examples of non-mammalian host cells and cell lines that may be used in accordance with the present invention include cells and cell lines derived from Pichia pastoris, Pichia methanolica, Pichia angusta, Schizosacccharomyces pombe, Saccharomyces cerevisiae, and Yarrowia lipolytica for yeast; Sodoptera frugiperda, Trichoplusis ni, Drosophila melangoster and Manduca sexta for insects; and Escherichia coli, Salmonella typhimurium, Bacillus subtilis, Bacillus lichemfonnis, Bacteroides fragilis, Clostridia perfringens, Clostridia difficile for bacteria; Xenopus Laevis from amphibian; and Daucus carota, tobacco Nicotiana tabacum, Zizania aquatic, Zizania palustris, Zizania latifolia and Lemna (duckweed) from plant.

### Large Scale Production of Highly Active I2S Fusion Protein

According to the present invention, cells engineered to express I2S immunoglobulin fusion protein are selected for their ability to produce the I2S immunoglobulin fusion protein at commercially viable scale. In particular, engineered cells according to the present invention are able to produce I2S fusion protein at a high level and with high enzymatic activity.

As discussed above, typically, the enzyme activity of I2S is influenced by a post-translational modification of a conserved cysteine (e.g., at amino acid 59) to formylglycine. This post-translational modification occurs in the endoplasmic reticulum during protein synthesis and is catalyzed by FGE. The enzyme activity of I2S is positively correlated with the extent to which the I2S has the formylglycine modification. For example, an I2S preparation that has a relatively high amount of formylglycine modification typically has a relatively high specific enzyme activity; whereas an I2S preparation that has a relatively low amount of formylglycine modification typically has a relatively low specific enzyme activity.

It is further contemplated that the intracellular ratio between the I2S and FGE protein or mRNA may also affect the extent of formylglycine modification on the produced I2S fusion protein. In some embodiments, the I2S and FGE expressed in a desired cell have different protein and/or mRNA expression levels. In some embodiments, the I2S fusion protein or mRNA expression level is at least 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8, 9, 10, 15, 20, 25, 20, 35, 30, 45, 40, 50, 60, 70, 80, 90 or 100-fold higher than the protein or mRNA level of FGE. In some embodiments the recombinant FGE protein or mRNA expression level is at least 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8, 9, or 10-fold higher than the protein or mRNA level of I2S fusion protein. Various methods for measuring mRNA or protein levels are known in the art and may be used to practice the present invention. Exemplary methods for measuring mRNA level include, but are not limited to, Northern blot, QRTPCR, RNA sequencing, and microarray. Exemplary methods for measuring protein level include, but are not limited to, ELISA, Western blot, Alpha Screen, ECL and label-free bio-layer.

Thus, in some embodiments, desirable cells, once cultivated under a cell culture condition (e.g., a standard large scale suspension or adherent culture condition), can produce I2S fusion protein with an average harvest titer (mg/L) of or greater than about 30 mg/L/day, 35 mg/L/day, 40 mg/L/day, 45 mg/L/day, 50 mg/L/day, 55 mg/L/day, 60 mg/L/day, 65 mg/L/day, 70 mg/L/day, 75 mg/L/day, 80 mg/L/day, 85 mg/L/day, 90 mg/L/day, 95 mg/L/day, 100 mg/L/day, 105 mg/L/day, 110 mg/L/day, 115 mg/L/day, 120 mg/L/day, 125 mg/L/day, 130 mg/L/day, 135 mg/L/day, 140 mg/L/day, 145 mg/L/day, 150 mg/L/day, 200 mg/L/day, 250 mg/L/day, 300 mg/L/day, 350 mg/L/day, 400 mg/L/day, 450 mg/L/day, 500 mg/L/day, 550 mg/L/day, 600 mg/L/day, or 650 mg/L/day. As used herein, the term “titer” refers to the total time average amount of recombinantly expressed polypeptide or protein produced daily by a cell culture divided by a given amount of medium volume.

In some embodiments, desirable cells, once cultivated under a cell culture condition (e.g., a standard large scale suspension or adherent culture condition), can produce I2S fusion protein in an amount of or greater than about 0.1 picogram/cell/day (e.g., greater than about 0.1, 0.15, 0.2, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 20, 25, 30, 35, 40, 45, 50, 55, 60, 65, 70, 75, 80, 85, 90, 95, or 100 picogram/cell/day). In some embodiments, desired cells, once cultivated under a cell culture condition (e.g., a standard large scale suspension or adherent culture condition), are able to produce I2S enzyme in an amount ranging from about 1-10 picogram/cell/day (e.g., about 1-9 picogram/cell/day, about 1-8 picogram/cell/day, about 1-7 picogram/cell/day, about 1-6 picogram/cell/day, about 1-5 picogram/cell/day, about 1-4 picogram/cell/day, about 1-3 picogram/cell/day, about 2-9 picogram/cell/day, about 2-8 picogram/cell/day, about 2-7 picogram/cell/day, about 2-6 picogram/cell/day, about 2-5 picogram/cell/day, about 2-4 picogram/cell/day, about 2-3 picogram/cell/day).

In some embodiments, desirable cells, once cultivated under a cell culture condition (e.g., a standard large scale suspension or adherent culture condition), can produce an I2S fusion protein comprising at least about 60% (e.g., at least about 65%, 70%, 75%, 80%, 85%, 90%, 95%, 96%, 97%, 98%, 99%, or 100%) conversion of the cysteine residue corresponding to Cys59 of SEQ ID NO:1 to Cα-formylglycine (FGly).

Various methods are known and can be used to determine the FGly conversion percentage. Generally, the percentage of formylglycine conversion (% FG) can be calculated using the following formula:

$$\% FG ( of DS ) = \frac{Number of active I 2 S molecules}{Number of total ( active + inactive ) I 2 S molecules} \times 100$$

For example 50% FG means half of the purified I2S fusion protein is enzymatically inactive without any therapeutic effect. Various methods may be used to calculate % FG. For example, peptide mapping may be used. Briefly, an 12S protein may be digested into short peptides using a protease (e.g., trypsin or chymotrypsin). Short peptides may be separated and characterized using chromatography (e.g., HPLC) such that the nature and quantity of each peptide (in particular the peptide containing the position corresponding to position 59 of the mature human 12S) may be determined, as compared to a control (e.g., an I2S protein without FGly conversion or an I2S protein with 100% FGly conversion). The amount of peptides containing FGly (corresponding to number of active I2S molecules) and the total amount of peptides with both FGly and Cys (corresponding to number of total I2S molecules) may be determined and the ratio reflecting % FG calculated.

### Cell Culture Medium and Condition

Various cell culture medium and conditions may be used to produce an I2S immunoglobulin fusion protein. For example, an I2S immunoglobulin fusion protein may be produced in serum-containing or serum-free medium. In some embodiments, an I2S immunoglobulin fusion protein is produced in chemically-defined medium. In some embodiments, an I2S immunoglobulin fusion protein is produced in an animal free medium, i.e., a medium that lacks animal-derived components. In some embodiments, an I2S immunoglobulin fusion protein is produced in a chemically defined medium. As used herein, the term “chemically-defined nutrient medium” refers to a medium of which substantially all of the chemical components are known. In some embodiments, a chemically defined nutrient medium is free of animal-derived components such as serum, serum derived proteins (e.g., albumin or fetuin), and other components. In some cases, a chemically-defined medium comprises one or more proteins (e.g., protein growth factors or cytokines.) In some cases, a chemically-defined nutrient medium comprises one or more protein hydrolysates. In other cases, a chemically-defined nutrient medium is a protein-free media, i.e., a serum-free media that contains no proteins, hydrolysates or components of unknown composition.

In some embodiments, a chemically defined medium may be supplemented by one or more animal derived components. Such animal derived components include, but are not limited to, fetal calf serum, horse serum, goat serum, donkey serum, human serum, and serum derived proteins such as albumins (e.g., bovine serum albumin or human serum albumin).

In some embodiments, the cells producing HIRMab-I2S fusion protein are cultured in a bioreactor. Various cell culture conditions may be used to produce I2S immunoglobulin fusion proteins at large scale including, but not limited to, roller bottle cultures, bioreactor batch cultures, bioreactor fed-batch cultures, bioreactor wave perfusion cultures, and bioreactor stirred tank perfusion cultures. In some embodiments, I2S fusion protein is produced by cells cultured in suspense. In some embodiments, I2S fusion protein is produced by adherent cells. In certain embodiments, the bioreactor operates as a stirred tank perfusion bioreactor process.

Exemplary cell media and culture conditions are described in the Examples sections.

### Purification of I2S Immunoglobulin Fusion Protein

In some embodiments, the present invention provides a method of purifying I2S immunoglobulin fusion protein from an impure preparation using a process based on one or more of affinity chromatography, cation-exchange chromatography, and mulitmodal chromatography. In some embodiments, an inventive method according to the present invention involves less than 4 (e.g., less than 4 or less than 3) chromatography steps. In some embodiments, an inventive method according to the present invention involves 2, 3, or 4 chromatography steps. In some embodiments, an inventive method according to the present invention involves 3 chromatography steps. In some embodiments, an inventive method according to the present invention conducts affinity chromatography, cation-exchange chromatography, and multimodal chromatography in that order.

### Impure Preparation

As used herein, an impure preparation can be any biological material including unprocessed biological material containing I2S immunoglobulin fusion protein. For example, an impure preparation may be unprocessed cell culture medium containing I2S immunoglobulin fusion protein secreted from the cells (e.g., mammalian cells) producing I2S immunoglobulin fusion protein or raw cell lysates containing I2S immunoglobulin fusion protein. In some embodiments, an impure preparation may be partially processed cell medium or cell lysates. For example, cell medium or cell lysates can be concentrated, diluted, treated with viral inactivation, viral processing or viral removal. In some embodiments, viral removal may utilize nanofiltration and/or chromatographic techniques, among others. In some embodiments, viral inactivation may utilize solvent inactivation, detergent inactivation, pasteurization, acidic pH inactivation, and/or ultraviolet inactivation, among others. In some embodiments, a low pH viral inactivation step occurs after the affinity column step and prior to the cation-exchange column step. In certain embodiments, the affinity chromatography eluate sample is held at low pH (e.g. about 3.6-3.8 pH) for about 30-60 minutes in order to inactivate enveloped viruses. In some embodiments, a viral filtration step occurs after the last chromatography column step is performed.

Cell medium or cell lysates may also be treated with protease, DNases, and/or RNases to reduce the level of host cell protein and/or nucleic acids (e.g., DNA or RNA). In some embodiments, unprocessed or partially processed biological materials (e.g., cell medium or cell lysate) may be frozen and stored at a desired temperature (e.g., 2-8° C., −4° C., −25° C., −75° C.) for a period time and then thawed for purification. As used herein, an impure preparation is also referred to as starting material or loading material.

### Affinity Chromatography

In some embodiments, provided methods for purifying I2S immunoglobulin fusion protein include affinity chromatography. In brief, affinity chromatography is a chromatographic technique which relies on highly specific interaction such as that between antigen and antibody, enzyme and substrate, or receptor and ligand, to separate biochemical mixtures. In some embodiments, the affinity chromatography is antigen and antibody chromatography, specifically Protein A chromatography.

Protein A affinity chromatography is generally practiced where target protein is adsorbed to Protein A immobilized on a solid phase comprising silica or glass; contaminants bound to the solid phase are removed by washing with a hydrophobic electrolyte solvent; and target protein is recovered from the solid phase. Suitable Protein A resins are known in the art and are commercially available and include, but are not limited to MabSelect SuRe®, Mab Select®, and Protein A Sepharose®. In certain embodiments, the Protein A affinity chromatography resin is a MabSelect SuRe® resin.

In certain embodiments, the affinity chromatography is practiced where the affinity chromatography column is eluted using an elution buffer comprising an isocratic Na Citrate elution. In some embodiments, the elution buffer comprises 25 mM, 30 mM, 35 mM, 40 mM, 45 mM, 50 mM, 55 mM, or 60 mM Na Citrate. In certain embodiments, the elution buffer comprises 50 mM Na Citrate. In certain embodiments, the Na Citrate isocratic elution comprises a range from 0-250 mM Na Citrate, 0-200 mM, 0-150 mM Na Citrate, 0-100 mM Na Citrate, 0-50 mM Na Citrate, or 0-25 mM Na Citrate.

In some embodiments, the elution buffer comprising an isocratic Na Citrate elution comprises a pH of 3.0, 3.1, 3.2, 3.3, 3.4, 3.5, 3.6, 3.7, 3.8, 3.9, or 4.0. In some embodiments, the pH of the elution buffer falls within a range of 3.0-4.0, 3.1-3.9, 3.2-3.8, 3.3-3.7, 3.4-3.6, or 3.6-3.7.

### Cation Exchange Chromatography

In some embodiments, provided methods for purifying I2S fusion protein include cation-exchange chromatography. In brief, cation exchange chromatography is a chromatographic technique which relies on charge-charge interactions between a positively charged compound and a negatively charged resin. In some embodiments, the cation-exchange chromatography is strong cation-exchange chromatography.

Cation exchange chromatography is generally practiced with either a strong or weak cation exchange column, containing a sulfonium ion, or with a weak cation exchanger, having usually a carboxymethyl (CM) or carboxylate (CX) functional group. Many suitable cation exchange resins are known in the art and are commercially available and include, but are not limited to Capto SP ImpRes®, SP-Sepharose®, CM Sepharose®; Amberjet® resins; Amberlyst® resins; Amberlite® resins (e.g., Amberlite® IRA120); ProPac® resins (e.g., ProPac® SCX-10, ProPac® WCX-10, ProPac® WCX-10); TSK-GEL® resins (e.g., TSKgel BioAssist S; TSKgel SP-2SW, TSKgel SP-SPW; TSKgel SP-NPR; TSKgel SCX; TSKgel SP-STAT; TSKgel CM-SPW; TSKgel OApak-A; TSKgel CM-2SW, TSKgel CM-3SW, and TSKgel CM-STAT); and Acclaim® resins. In certain embodiments, the cation exchange resin is Capto SP ImpRes®.

In some embodiments, the cation-exchage chromatography column is eluted using an elution buffer comprising an isocratic NaCl elution. In certain embodiments, the elution buffer comprises a range from 0-400 mM NaCl, 0-350 mM NaCl, 0-300 mM NaCl, 0-250 mM NaCl, or 0-200 mM NaCl.

Typically, the isocratic elution is buffered. In certain embodiments, the isocratic elution is not buffered. In certain embodiments, the isocratic elution is buffered to a pH between about 5 to about 14. In certain embodiments, the isocratic elution is buffered to a pH between about 5 to about 10. In certain embodiments, the isocratic elution is buffered to a pH between about 5 to about 7. In certain embodiments, the isocratic elution is buffered to a pH between about 5.5 to about 6.0. In certain embodiments, the isocratic elution is buffered to a pH between about 5.2 to about 5.8. In certain embodiments, the cation-exchange chromatography column is run at a pH of between 5.2 and 5.8. In certain embodiments, the isocratic elution is buffered to a pH of about 5.0, 5.5, 6.0, 6.5, 7.0, 7.5, 8.0, 8.5, 9.0, 9.5, or 10.

### Multimodal Chromatography

In some embodiments, provided methods for purifying I2S immunoglobulin fusion protein include multimodal chromatography. In brief, multimodal chromatography is a chromatographic technique which provides intermediate purification by relying on ion exchange interactions between intermediately charged compounds. The target protein can flow through the column, while the impurities (e.g. host-cell proteins (HCP)) bind to the column. In some embodiments, the multimodal chromatography resin is a Capto Adhere resin. In some embodiments, the multimodal chromatography column is operated in flow through mode. In certain embodiments, the multimodal chromatography column is operated in bind/elute mode. In some embodiments, the multimodal chromatography is operated in a combination of flow through mode and bind/elute mode.

In some embodiments, the multimodal chromatography column is run at a pH between about 4.5 to about 7.5, between about 5.0 to about 7.0, or between about 5.5 to about 6.5. In certain embodiments, the multimodal chromatography column is run at a pH of about 7.0.

In some embodiments, a loading and washing is performed at the beginning and/or throughout the purification process. In certain embodiments, a salt concentration of between about 1.0M and about 2.0M NaCl was used in loading and washing the chromatography columns.

### Characterization of I2S Immunoglobulin Fusion Proteins

Purified I2S immunoglobulin proteins may be characterized using various methods.

### HIR Binding Assay; BBB Transport

In certain embodiments, the immunoglobulin facilitates at least about 70% binding to Human Insulin receptors, 80% binding to Human Insulin receptors, 90% binding to Human Insulin receptors, or 95% binding to Human Insulin receptors.

### M6P Binding Assay; Lysosomal Transport

In some embodiments, 2-M6P residues present on the I2S bind to M6P receptors and mediate transport via endogenous lysosomal M6P receptors. In certain embodiments, the 2-M6P residues facilitate at least about 60% binding to M6P receptors, at least about 70% binding to M6P receptors, or at least about 75% binding to M6P receptors.

In some embodiments, the HIRMab-I2S fusion protein comprises between 1% and 9% 2-mannose-6-phosphate (2-M6P) peak area on a glycan map, which is measured via a 2-aminobenzamide glycan labelling process. In certain embodiments, the HIRMab-I2S fusion protein comprises between 5% and 8% 2-mannose-6-phosphate (2-M6P) peak area on a glycan map, or between 5.2% and 7.2% 2-mannose-6-phosphate (2-M6P) peak area on a glycan map. In certain embodiments, the HIRMab-I2S fusion protein comprises between 5.2% and 7.2% 2-mannose-6-phosphate (2-M6P) residue levels.

### Purity

The purity of purified I2S immunoglobulin fusion protein is typically measured by the level of various impurities (e.g., host cell protein or host cell DNA) present in the final product. For example, the level of host cell protein (HCP) may be measured by ELISA or SDS-PAGE. In some embodiments, the purified HIRMab-I2S fusion protein contains less than 10 ng HCP/mg I2S fusion protein (e.g., less than 9, 8, 7, 6, 5, 4, 3 ng HCP/mg I2S fusion protein). Various assay controls may be used, in particular, those acceptable to regulatory agencies such as FDA.

### Specific Activity—4-MU Assay

In some embodiments, the enzymatic activity of I2S immunoglobulin fusion protein may be determined using various methods known in the art such as, for example, 4-MU assay which measures hydrolysis of 4-methylumbelliferyl sulfate (4-MUS) to sulfate and naturally fluorescent 4-methylumbelliferone (4-MU). In some embodiments, a desired enzymatic activity, as measured by in vitro 4-MU assay, of the produced I2S immunoglobulin fusion protein is at least about 0.5 U/mg, 1.0 U/mg, 1.5 U/mg, 2 U/mg, 2.5 U/mg, 3 U/mg, 4 U/mg, 4.5 U/mg, or 5.0 U/mg. Exemplary conditions for performing in vitro 4-MU assay are provided below. Typically, a 4-MU assay measures the ability of an I2S protein to hydrolyze 4-methylumbelliferyl sulfate (4-MUS) to sulfate and naturally fluorescent 4-methylumbelliferone (4-MU). One milliunit of activity is defined as the quantity of enzyme required to convert one nanomole of 4-MUS to 4-MU in one minute at 37° C. Typically, the mean fluorescence units (MFU) generated by I2S test samples with known activity can be used to generate a standard curve, which can be used to calculate the enzymatic activity of a sample of interest. Specific activity may then calculated by dividing the enzyme activity by the protein concentration.

In some embodiments, specific activity is measured using a plate-based fluorometric enzyme activity assay which measures the hydrolysis of 4-methylumbelliferyl sulfate (4-MUS) to sulfate and 4-methylumbelliferone (4-MU). In such embodiments, samples are incubated with the 4-MUS substrate solution at 37° C., pH 5.0, for 60 min in a 96-well plate. The enzymatic reaction can then be stopped by the addition of glycine carbonate stop buffer at pH 10.7. The high pH also generates the fluorescent, anionic form of the 4-MU product, which can be measured at excitation and emission wavelengths of 360 nm and 460 nm, respectively. The amount of 4-MU generated in the enzyme-catalyzed reaction can be interpolated from a 4-MU standard curve.

The reportable values can be expressed in U/mg of DS, where U is defined as the quantity of enzyme required to release one micromole of 4-methylumbelliferone per minute at 37° C. and pH 5.0.

In either example, the protein concentration of an I2S immunoglobulin fusion protein composition may be determined by any suitable method known in the art for determining protein concentrations. In some cases, the protein concentration is determined by an ultraviolet light absorbance assay. In some embodiments, such absorbance assays are measured at excitation and emission wavelengths of 360 nm and 460 nm, respectively.

### Specific Activity—IdoA2S-4-MU Assay

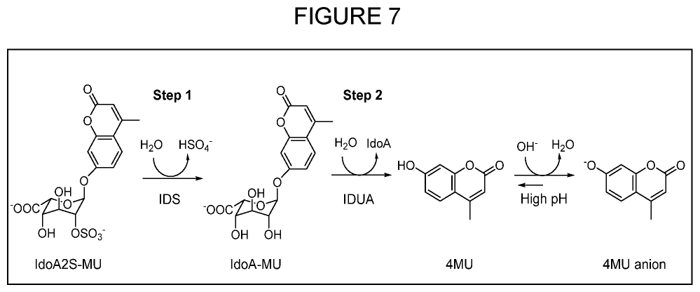

In some embodiments, the enzymatic activity of I2S immunoglobulin fusion protein may be determined using various methods known in the art such as, for example, IdoA2S-4-MU assay, a schematic of which is depicted in FIG. 7. In some embodiments, a desired enzymatic activity, as measured by in vitro IdoA2S-4-MU assay, of the produced I2S immunoglobulin fusion protein is at least about 1 U/mg, 2.5 U/mg, 5 U/mg, 10 U/mg, 15 U/mg, 20 U/mg, 25 U/mg, 30 U/mg, 35 U/mg, 40 U/mg, 45 U/mg, 50 U/mg, 55 U/mg, 60 U/mg, 65 U/mg, or 70 U/mg. Exemplary conditions for performing in vitro IdoA2S-4-MU assay are provided below. Typically, an IdoA2S-4-MU assay measures the ability of an I2S protein via a two-step method. In the first step; the substrate IdoA2S-4-MU is hydrolyzed to umbelliferyl-α-L-idopyranosiduronic acid (IdoA-4-MU) and sulfate. In the second step, a complete conversion of IdoA-4-MU to naturally fluorescent 4-MU can be achieved by the addition of excess amount of IDUA. Typically, the mean fluorescence units (MFU) generated by 12S test samples with known activity can be used to generate a standard curve, which can be used to calculate the enzymatic activity of a sample of interest. Specific activity may then calculated by dividing the enzyme activity by the protein concentration.

In some embodiments, specific activity is measured using a plate-based fluorometric enzyme activity assay. In some embodiments, the reaction can be carried out in 96-well PCR plate with a temperature controlled thermocycler. In such embodiments, the reaction can be initiated by mixing 20 μL each of 2 mM IdoA2S-4-MU substrate solution and 5 ng/mL I2S sample solution in 2× assay buffer, which will be incubated for one hour at 37° C. In some embodiments, the buffer can comprise a 50 mM acetate-buffered reaction mixture, pH 5.2, containing 0.03 mg/mL of BSA. In some embodiments, 40 μL of 25 μg/mL IDUA in McIlvaine's buffer (0.40 M sodium phosphate, 0.20 M citrate, 0.02% sodium azide, pH 4.5) can be added to arrest the I2S reaction, which can be incubated for an additional hour at the same temperature. In some embodiments, the second step reaction can be quenched by addition of 200 μL of 0.5 M sodium carbonate solution, pH 10.7. In such embodiments, the observed fluorescence of 4-MU can be measured at λₑₓ and λₑₘ of 365 and 450 nm, respectively.

### Glycan Mapping

In some embodiments, a purified I2S fusion protein may be characterized by its proteoglycan composition, typically referred to as glycan mapping. Without wishing to be bound by any theory, it is thought that glycan linkage along with the shape and complexity of the branch structure may impact in vivo clearance, lysosomal targeting, bioavailability, and/or efficacy.

Typically, a glycan map may be determined by enzymatic digestion and subsequent chromatographic analysis. Various enzymes may be used for enzymatic digestion including, but not limited to, suitable glycosylases, peptidases (e.g., Endopeptidases, Exopeptidases), proteases, and phosphatases. In some embodiments, a suitable enzyme is alkaline phosphatase. In some embodiments, a suitable enzyme is neuraminidase. Glycans (e.g., phosphoglycans) may be detected by chromatographic analysis. For example, phosphoglycans may be detected by High Performance Anion Exchange Chromatography with Pulsed Amperometric Detection (HPAE-PAD) or size exclusion High Performance Liquid Chromatography (HPLC). The quantity of glycan (e.g., phosphoglycan) represented by each peak on a glycan map may be calculated using a standard curve of glycan (e.g., phosphoglycan), according to methods known in the art and disclosed herein.

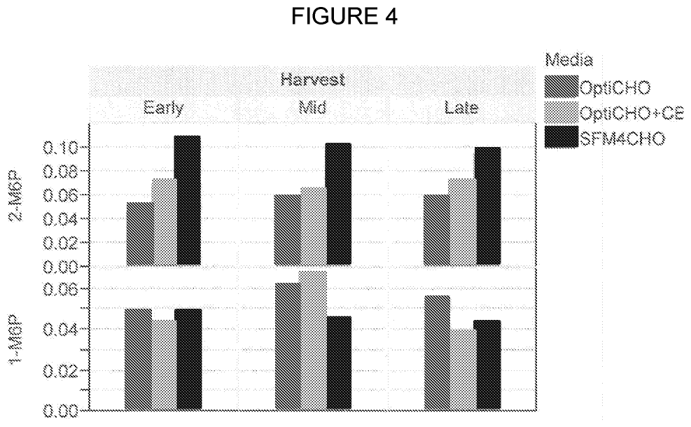

In some embodiments, a purified I2S fusion protein according to the present invention exhibits a glycan map comprising eight peak groups indicative of neutral (peak group 1), mono-sialylated (peak group 2), di-sialylated (peak group 3), monophosphorylated (peak group 4), tri-sialylated (peak group 5), tetra-sialylated (peak group 6), diphosphorylated (peak group 7), and peak group 8 I2S fusion protein, respectively. Exemplary analyses of glycan content of I2S fusion protein are depicted in FIGS. 4, 5, and 6. In some embodiments, a purified I2S immunoglobulin fusion protein has a glycan map that has fewer than 8 peak groups (e.g., a glycan map with 7, 6, 5, 4, 3, or 2 peaks groups). In some embodiments, a purified I2S fusion protein has a glycan map that has more than 8 peak groups (e.g., 9, 10, 11, 12 or more).

The relative amount of glycan corresponding to each peak group may be determined based on the peak group area relative to the corresponding peak group area in a predetermined reference standard. In some embodiments, peak group 1 (neutral) may have the peak group area ranging from about 40-120% (e.g., about 40-115%, about 40-110%, about 40-100%, about 45-120%, about 45-115%, about 45-110%, about 45-105%, about 45-100%, about 50-120%, about 50-110%) relative to the corresponding peak group area in a reference standard. In some embodiments, peak group 2 (monosialylated) may have the peak group area ranging from about 80-140% (e.g., about 80-135%, about 80-130%, about 80-125%, about 90-140%, about 90-135%, about 90-130%, about 90-120%, about 100-140%) relative to the corresponding peak group area in the reference standard. In some embodiments, peak group 3 (disialylated) may have the peak group area ranging from about 80-110% (e.g., about 80-105%, about 80-100%, about 85-105%, about 85-100%) relative to the corresponding peak group area in the reference standard. In some embodiments, peak group 4 (monophosphorylated) may have the peak group area ranging from about 100-550% (e.g., about 100-525%, about 100-500%, about 100-450%, about 150-550%, about 150-500%, about 150-450%, about 200-550%, about 200-500%, about 200-450%, about 250-550%, about 250-500%, about 250-450%, or about 250-400%) relative to the corresponding peak group area in the reference standard. In some embodiments, peak group 5 (tri-sialylated) may have the peak group area ranging from about 70-110% (e.g., about 70-105%, about 70-100%, about 70-95%, about 70-90%, about 80-110%, about 80-105%, about 80-100%, or about 80-95%) relative to the corresponding peak group area in the reference standard. In some embodiments, peak group 6 (tetra-sialylated) may have the peak group area ranging from about 90-130% (e.g., about 90-125%, about 90-120%, about 90-115%, about 90-110%, about 100-130%, about 100-125%, or about 100-120%) relative to the corresponding peak group area in the reference standard. In some embodiments, peak group 7 (diphosphorylated) may have with the peak group area ranging from about 70-130% (e.g., about 70-125%, about 70-120%, about 70-115%, about 70-110%, about 80-130%, about 80-125%, about 80-120%, about 80-115%, about 80-110%, about 90-130%, about 90-125%, about 90-120%, about 90-115%, about 90-110%) relative to the corresponding peak group area in the reference standard. Various reference standards for glycan mapping are known in the art and can be used to practice the present invention. Typically, peak group 7 (diphosphorylated) corresponds to the level of di-M6P on the surface of the purified I2S fusion protein.

It is contemplated that the glycosylation pattern of a purified I2S impacts the lysosomal and neuronal membrane targeting. Various in vitro cellular uptake assays are known in the art and can be used to practice the present invention. For example, to evaluate the uptake of I2S by M6P receptors, cellular uptake assays are performed using human fibroblasts expressing M6P receptors on their surface. The internalized amount of I2S can be measured by a ELISA method. In some embodiments, a purified I2S fusion protein according to the present invention is characterized with cellular uptake of greater than 70%, 75%, 80%, 85%, 90%, 95%, as determined by an in vitro uptake assay.

### Percent Formylglycine Conversion

Peptide mapping can be used to determine Percent FGly conversion. As discussed above, I2S activation requires Cysteine (corresponding to position 59 of the mature human I2S) to formylglycine conversion by formylglycine generating enzyme (FGE) as shown below:

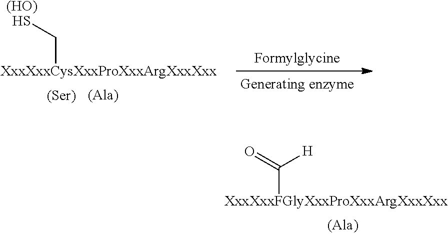

Therefore, the percentage of formylglycine conversion (% FG) can be calculated using the following formula:

$$\% FG ( of DS ) = \frac{Number of active I 2 S molecules}{Number of total ( active + inactive ) I 2 S molecules} \times 100$$

To calculate % FG, an I2S immunoglobulin fusion protein may be digested into short peptides using a protease (e.g., trypsin or chymotrypsin). Short peptides may be separated and characterized using, e.g., size exclusion High Performance Liquid Chromatography (HPLC). The peptide containing the position corresponding to position 59 of the mature human I2S may be characterized to determine if the Cys at position 59 was converted to a FGly as compared to a control (e.g., an I2S protein without FGly conversion or an I2S protein with 100% FGly conversion). The amount of peptides containing FGly (corresponding to number of active I2S molecules) and the total amount of peptides with both FGly and Cys (corresponding to number of total I2S molecules) may be determined based on the corresponding peak areas and the ratio reflecting % FG can be calculated.

In some embodiments, a purified I2S fusion protein according to the present invention has at least about 60% (e.g., at least about 60%, 65%, 70%, 75%, 80%, 85%, 90%, 95%, 96%, 97%, 98%, 99%) conversion of the cysteine residue corresponding to Cys59 of human I2S (SEQ ID NO:1) to C_{α}-formylglycine (FGly). In some embodiments, a purified I2S fusion protein according to the present invention has substantially 100% conversion of the cysteine residue corresponding to Cys59 of human I2S (SEQ ID NO:1) to C_{α}-formylglycine (FGly).

### Sialic Acid Content

In some embodiments, a purified I2S fusion protein may be characterized by its sialic acid composition. Without wishing to be bound by theory, it is contemplated that sialic acid residues on proteins may prevent, reduce or inhibit their rapid in vivo clearance via the asialoglycoprotein receptors that are present on hepatocytes. Thus, it is thought that 12S fusion proteins that have relatively high sialic acid content typically have a relatively long circulation time in vivo.

In some embodiments, the sialic acid content of a purified 12S fusion protein may be determined using methods well known in the art. For example, the sialic acid content of an 12S fusion protein may be determined by enzymatic digestion and subsequent chromatographic analysis. Enzymatic digestion may be accomplished using any suitable sialidase. In some cases, the digestion is performed by a glycoside hydrolase enzyme, such as neuraminidase. Sialic acid may be detected by chromatographic analysis such as, for example, High Performance Anion Exchange Chromatography with Pulsed Amperometric Detection (HPAE-PAD). The quantity of sialic acid in a purified I2S fusion protein composition may be calculated using a standard curve of sialic acid, according to methods known in the art and disclosed herein.

In some embodiments, the sialic acid content of a purified I2S fusion protein may be at least 15 mol/mol. In some embodiments, the sialic acid content of a purified I2S fusion protein may be at least 20 mol/mol. The units “mol/mol” in the context of sialic acid content refers to moles of sialic acid residue per mole of enzyme. In some cases, the sialic acid content of an I2S immunoglobulin fusion protein is or greater than about 16.5 mol/mol, about 17 mol/mol, about 18 mol/mol, about 19 mol/mol, about 20 mol/mol, about 21 mol/mol, about 22 mol/mol, about 23 mol/mol, or more. In some embodiments, the sialic acid content of a purified I2S immunoglobulin fusion protein may be in a range between about 17-20 mol/mol, 17-21 mol/mol, about 17-22 mol/mol, 17-23 mol/mol, 17-24 mol/mol, about 17-25 mol/mol, about 18-20 mol/mol, 18-21 mol/mol, about 18-22 mol/mol, 18-23 mol/mol, 18-24 mol/mol, or about 18-25 mol/mol.

### Pharmaceutical Composition and Administration

I2S fusion protein according to the present invention may be used to treat a subject who is susceptible to or suffering from I2S deficiency (e.g. Hunter syndrome). The present invention is particularly useful for treatment of I2S deficiency in the CNS, wherein direct administration into the CNS involves physical penetration or disruption of the BBB. Because of its ability to cross the BBB via receptor-mediated transport, some embodiments of the present invention provide for systemic administration of a pharmaceutical composition comprising the HIRMab-I2S fusion protein. Systemic administration routes include, but are not limited to, intravenous, intra-arterial intramuscular, subcutaneous, intraperitoneal, intranasal, transbuccal, transdermal, rectal, transalveolar (inhalation), or oral administration. In some embodiments, systemic administration of an I2S fusion protein described herein may be performed in combination with other direct CNS administration such as intrathecal delivery. In some embodiments, the pharmaceutical composition comprising HIRMab-I2S fusion protein can treat both somatic and cognitive symptoms of I2S deficiency.

An I2S deficiency as referred to herein includes, one or more conditions known as Hunter syndrome, Hunter disease, and mucopolysaccharidosis type II. The I2S deficiency is characterized by the buildup of heparin sulfate and dermatan sulfate that occurs in the body (the heart, liver, brain etc.).

In some embodiments, a HIRMab-I2S fusion protein or a pharmaceutical composition containing the same is administered to a subject by intravenous administration. In embodiments, the pharmaceutical composition comprises an HIRMab-I2S fusion protein that has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:11. In embodiments, the pharmaceutical composition comprises an HIRMab-I2S fusion protein that has an amino acid sequence identical to SEQ ID NO: 11. In embodiments, the pharmaceutical composition comprises an HIRMab-I2S fusion protein that has a recombinant human IgG light chain. In embodiments, the recombinant human IgG light chain has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:12. In embodiments, the the recombinant human IgG light chain has an amino acid sequence identical to SEQ ID NO: 12. In some embodiments, a HIRMab-I2S fusion protein or a pharmaceutical composition containing the same is administered to the subject by subcutaneous (i.e., beneath the skin) administration. For such purposes, the formulation may be injected using a syringe. However, other devices for administration of the formulation are available such as injection devices (e.g., the Inject-ease™ and Genject™ devices); injector pens (such as the GenPen™); needleless devices (e.g., MediJector™ and BioJector™); and subcutaneous patch delivery systems.

The present invention contemplates single as well as multiple administrations of a therapeutically effective amount of a HIRMab-I2S fusion protein or a pharmaceutical composition containing the same described herein. A HIRMab-I2S fusion protein or a pharmaceutical composition containing the same can be administered at regular intervals, depending on the nature, severity and extent of the subject's condition. In some embodiments, a therapeutically effective amount of a HIRMab-I2S fusion protein or a pharmaceutical composition containing the same may be administered periodically at regular intervals (e.g., once every year, once every six months, once every five months, once every three months, bimonthly (once every two months), monthly (once every month), biweekly (once every two weeks), weekly, daily or continuously).

A HIRMab-I2S fusion protein or a pharmaceutical composition containing the same can be formulated with a physiologically acceptable carrier or excipient to prepare a pharmaceutical composition. The carrier and therapeutic agent can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In some embodiments, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition). For example, a therapeutically effective amount may be an amount sufficient to achieve a desired therapeutic and/or prophylactic effect, such as an amount sufficient to modulate lysosomal enzyme receptors or their activity to thereby treat such lysosomal storage disease or the symptoms thereof (e.g., a reduction in or elimination of the presence or incidence of “zebra bodies” or cellular vacuolization following the administration of the compositions of the present invention to a subject). Generally, the amount of a therapeutic agent (e.g., a recombinant lysosomal enzyme) administered to a subject in need thereof will depend upon the characteristics of the subject. Such characteristics include the condition, disease severity, general health, age, sex, and body weight of the subject. One of ordinary skill in the art will be readily able to determine appropriate dosages depending on these and other related factors. In addition, both objective and subjective assays may optionally be employed to identify optimal dosage ranges.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

It is to be further understood that for any particular subject, specific dosage regimens should be adjusted over time according to the individual need and the professional judgment of the person administering or supervising the administration of the enzyme replacement therapy and that dosage ranges set forth herein are exemplary only and are not intended to limit the scope or practice of the claimed invention.

In some embodiments, the compositions of the invention, e.g., a HIRMab-I2S fusion protein, may be administered as part of a combination therapy. The combination therapy involves the administration of a composition of the invention in combination with another therapy for treatment or relief of symptoms typically found in a patient suffering from an I2S deficiency. If the composition of the invention is used in combination with another CNS disorder method or composition, any combination of the composition of the invention and the additional method or composition may be used. Thus, for example, if use of a composition of the invention is in combination with another CNS disorder treatment agent, the two may be administered simultaneously, consecutively, in overlapping durations, in similar, the same, or different frequencies, etc. In some cases a composition will be used that contains a composition of the invention in combination with one or more other CNS disorder treatment agents.

In some embodiments, the composition, e.g., a HIRMab-I2S, is co-administered to the patient with another medication, either within the same formulation or as a separate composition. For example, a HIRMab-I2S may be formulated with another fusion protein that is also designed to deliver across the human blood-brain barrier a recombinant protein other than I2S. Further, the I2S fusion protein may be formulated in combination with other large or small molecules.

Additional exemplary pharmaceutical compositions and administration methods are described in PCT Publication WO2011/163649 entitled “Methods and Compositions for CNS Delivery of Iduronate-2-Sulfatase;” and provisional application Ser. No. 61/618,638 entitled “Subcutaneous administration of iduronate 2 sulfatase” filed on Mar. 30, 2012, the entire disclosures of both of which are hereby incorporated by reference.

It is to be further understood that for any particular subject, specific dosage regimens should be adjusted over time according to the individual need and the professional judgment of the person administering or supervising the administration of the enzyme replacement therapy and that dosage ranges set forth herein are exemplary only and are not intended to limit the scope or practice of the claimed invention.

Any of the above aspects and embodiments can be combined with any other aspect or embodiment as disclosed in the Drawings, in the Summary, and/or in the Detailed Description, including the below Examples.

### Examples

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1: HIR Mab-I2S Fusion Protein Capture and Purification Process

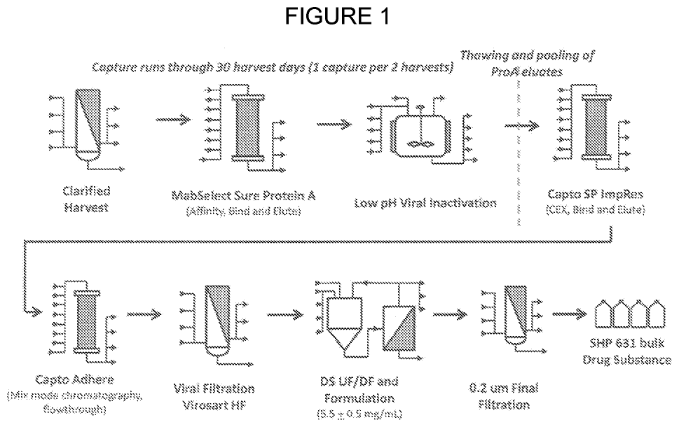

This example demonstrates a simplified downstream purification process that may be used to capture and purify HIR Mab-I2S fusion protein. An exemplary purification scheme is depicted in FIG. 1.

A cell line stably expressing a Human Insulin Receptor Monoclonal antibody-iduronate-2-sulfatase (HIRMab-I2S) fusion protein was developed. The HIRMab-I2S amino acid sequence is shown in Table 2. Generation and characterization of exemplary cell lines are described in the U.S. Pat. No. 8,834,874, entitled “Methods and Compositions for Increasing Iduronate-2-sulfatase Activity in the CNS” filed on Oct. 8, 2010, the entire contents of which is hereby incorporated by reference.

A stirred tank perfusion bioreactor process was used. A chemically defined media (CD OptiCHO) was used in the bioreactor process to increase cell density, higher viability, higher overall yield, and consistent product quality over 30 harvest days.

The downstream purification process began with clarified harvest material that is then purified by MabSelect Sure Protein A affinity chromatography. After a low pH viral inactivation step, thawing and pooling of the Protein A affinity chromatography eluates occurred. The purification process proceeded with successive steps of cation exchange (Capto SP ImpRes) and mixed mode (Capto Adhere) chromatography steps, followed by viral removal filtration and drug substance ultrafiltration/diafiltration and formulation at 5.0±0.5 mg/mL. A final filtration step was performed to produce the bulk drug substance. In particular, this purification process utilized Protein A affinity, Capto SP ImpRes, and Capto Adhere chromatographic modalities. Exemplary steps are shown in Table 3.

TABLE 3
Exemplary Steps of Purification Process

Clarified Harvest
Protein A Affinity Chromatography
Low pH Viral Inactivation
Frozen Storage
Thaw, Pool of ProA eluates
↓
Cation-Exchange Chromatography
↓
Mixed-mode Chromatography
↓
Viral Removal Filtration
↓
UF/DF and Formulation
↓
Final Filtration
↓
Drug Substance

### Example 2: Analysis of Mock DS HIR Mab-I2S Fusion Protein

Purified mock drug substance HIRMab-I2S fusion protein was assessed for purity by CHO HCP content, CE-SDS (non-reduced), and size exclusion chromatography (SEC %). Enzyme specific activity, sialic acid content, and glycan map were determined using standard methods. A substrate clearance assay was performed by measuring % RP. Exemplary results are shown in Table 4.

TABLE 4
Analysis of Mock DS Purified HIRMab-I2S Fusion Protein

| | | Purified HIRMab-I2S |
|---|---|---|
| | | Fusion Protein |
| Assay | | Min-Max (n) |
| Host Cell Protein | | <3-<8 (n = 4) |
| (ppm) | | |
| CE-SDS | | 99.03-99.83 (n = 4) |
| (non-reduced) | | |
| Size Exclusion | | 98.5-99.7% (n = 4) |
| Chromatography | | |
| (%) | | |
| Specific Activity | | 2.26-3.31 (n = 4) |
| (U/mg) | | |
| Glycan Map | Neutral | 17.53-24.66% (n = 4) |
| (Peak Group as | 1-SA | 10.13-12.30% (n = 4) |
| Percent Area) | 2-SA | 13.92-14.83% (n = 4) |
| | 1-M6P | 3.43-4.12% (n = 4) |
| | 3-SA | 16.34-19.46% (n = 4) |
| | 4-SA | 16.49-22.60% (n = 4) |
| | 2-M6P | 4.93-5.60% (n = 4) |
| | Peak 8 | 5.99-8.10% (n = 4) |
| Sialic Acid | | 18.26-22.62 (n = 4) |
| (mol/mol) | | |
| Substrate | | 79.5-94.1% (n = 3) |
| Clearance Assay | | |
| (% RP) | | |

In particular, the CE-SDS assay evaluates the purity/impurity profile and the ratio between antibody heavy and light chains. Specific activity was obtained using a plate-based fluorometric enzyme activity assay that measures the hydrolysis of 4-methylumbelliferyl sulfate (4-MUS) to sulfate and 4-methylumbelliferone (4-MU), wherein the 4-MU was measured as fluorescence at excitation and emission wavelengths of 360 nm and 460 nm, respectively, and the amount of 4-MU generated in the catalyzed reaction was interpolated from a 4-MU standard curve. Specific activity reportable values were expressed in U/mg, where U is defined as the quantity of enzyme required to release one micromole of 4-MU per minute at 37° C. and pH 5.0. The substrate clearance assay measured a cellular uptake and enzyme specific activity. The glycan map of purified HIRMab-I2S fusion protein includes seven peak groups, eluting according to an increasing amount of negative charges derived from sialic acid and mannose-6-phosphate residues, representing in the order of elution, neutrals, mono-, disialylated, monophosphorylated, trisialylated and hybrid (monosialylated and capped M6P), tetrasialylated and hybrid (disialylated and capped M6P) and diphosphorylated glycans.

Taken together, this example demonstrates that a simplified three-column purification process can be used to successfully purify HIRMab-I2S fusion protein produced in chemically-defined medium at large scale.

### Example 3: Exemplary HIR Mab-I2S Fusion Protein Product Quality

HIRMab-I2S fusion protein was produced by a process using a stirred tank, perfusion bioreactor at scale of 10 L. A chemically-defined media, OptiCHO plus Cell boost 5, was used. HIRMab-I2S fusion protein was purified by a process as described in Example 1.

Purified HIRMab-I2S fusion protein was assessed for formylglycine content, binding to the Human insulin receptor (% RS), Glycan map (1-M6P and 2-M6P, % peak areas), binding to M6P receptor (% RS), and Substrate Clearance assay (% to RS). Exemplary results are shown in Table 5.

TABLE 5
Exemplary Purified HIRMab-I2S Fusion Protein

| | Purified HIRMab-I2S |
|---|---|
| | Fusion Protein |
| | (10 L scale) |
| Assay | Min-Max (n = 3) |
| % Formylglycine | 98-100% |
| Human Insulin | 95% |
| receptor (% RS) | |
| Glycan Map | 3.7-6.7% |
| (1-M6P, % peak area) | |
| Glycan Map | 5.2-7.2% |
| (2-M6P, % peak area) | |
| M6P receptor | 75% |
| binding (% RS) | |
| Substrate Clearance | 97-108% |
| Assay (% to RS) | |

The process described herein consists of a Substrate Clearance Assay that measures a combination of cellular uptake and enzyme specific activity Percent Formylglycine Conversion

Peptide mapping can be used to determine Percent FGly conversion. I2S activation requires Cysteine (corresponding to position 59 of the mature human I2S) to formylglycine conversion by formylglycine generating enzyme (FGE) as shown below:

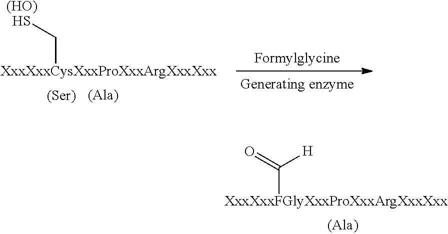

Therefore, the percentage of formylglycine conversion (% FG) can be calculated using the following formula:

$$\% FG ( of DS ) = \frac{Number of active I 2 S molecules}{Number of total ( active + inactive ) I 2 S molecules} \times 100$$

For example 50% FG means half of the HIRMab-I2S fusion protein is enzymatically inactive without any therapeutic effect.

Peptide mapping was used to calculate % FG. Briefly, HIRMab-I2S fusion protein was digested into short peptides using a protease (e.g., trypsin or chymotrypsin). Short peptides were separated and characterized using HPLC. The peptide containing the position corresponding to position 59 of the mature human I2S was characterized to determine if the Cys at position 59 was converted to a FGly as compared to a control (e.g., an I2S protein without FGly conversion or an I2S protein with 100% FGly conversion). The amount of peptides containing FGly (corresponding to number of active I2S molecules) and the total amount of peptides with both FGly and Cys (corresponding to number of total I2S molecules) may be determined based on the corresponding peak areas and the ratio reflecting % FG was calculated.

Table 6 shows a comparison of HIRMab-I2S fusion protein that was produced by a 10 L stirred tank, perfusion bioreactor process, using a chemically defined cell culture media (OptiCHO+CBS), and lots A1-A6 and larger scale lots that were produced using WAVE bioreactor with the addition of SFM4CHO (production media containing hydrolysates and animal components).

TABLE 6
Comparison between Lots A1-A6, B and C

| | Lot A1-A6 | Lot B | Lot C |
|---|---|---|---|
| % FG | 64-90% | 81% | 98-100% |
| Human Insulin | 93-101% | 110% | 95%² |
| receptor (% RS¹) | | | |
| Glycan map | 3.0-3.9% | 0.3% | 3.7-6.7% |
| (1-M6P, % peak area) | | | |
| Glycan map | 9.7-13.0% | 3.0% | 5.2-7.2% |
| (2-M6P, % peak area) | | | |
| M6P receptor | NT | <5% | 75%² |
| binding (% RS¹) | | (negligible) | |
| Substrate Clearance | 68-105% | 24% | 97-108% |
| Assay | | | |

¹MCB comparability lot ER2 lot serves as the assay reference standard (RS)
²n = 1, 1 BR tested
NT: Not tested

Typically, using a process described herein, the 2-M6P content was slightly lower than that measured in lots A1-A6, and the formylglycine content (% FGly) was higher. Taken together, this example demonstrates that a simplified three-column purification process can be used to successfully purify HIRMab-I2S fusion protein produced in chemically-defined medium at large scale with modulated levels of 2-M6P as compared to Iota A1-A6 lots.

### Example 4: Analysis of Media on HIRMab-I2S Fusion Protein Product Quality

The objective of this study was to assess media conditions and evaluate the effectiveness of protein production of HIRMab-I2S fusion protein in an animal-free perfusion process using chemically defined media (OptiCHO) with and without Cell Boost 5, and to characterize the product quality as compared to media conditions containing hydrolysates and animal components (SFM4CHO).

This study evaluated HIRMab-I2S production process performance and product quality obtained from a chemically defined medium bioreactor.

HIRMab-I2S fusion protein harvest samples from early, middle, and late harvest stages were pooled and captured. Harvest material was produced from a cell line using a perfusion wave 10 L bioreactor with a centrifuge retention device and using a chemically defined expansion media (OptiCHO) that included the additive Cell boost 5. Harvest material was produced from a cell line using a perfusion wave 10 L bioreactor with a centrifuge device and using a chemically defined expansion media that did not include the additive Cell boost 5. Harvest material was also produced from a cell line using a centrifuge perfusion process with no bleeding and using SFM4CHO media containing hydrolysates and animal components.

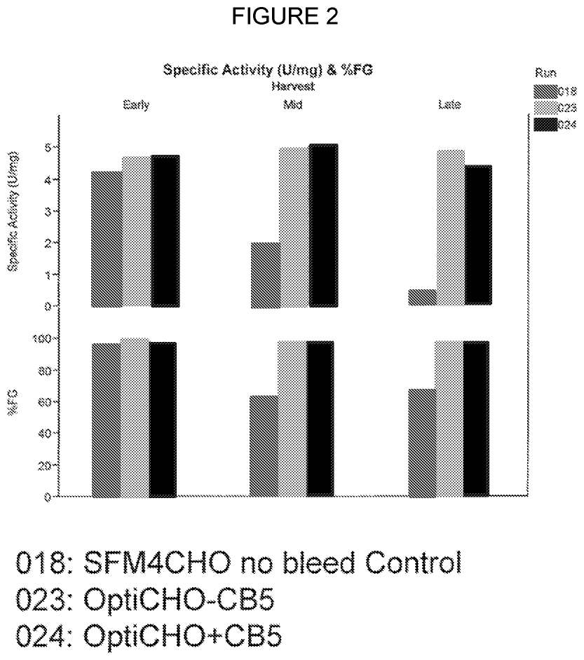

FIG. 2 demonstrates the specific activity (U/mg) and formylglycine content (% FG) of early, mid, and late HIRMab-I2S fusion protein harvest materials obtained under aforementioned media conditions.

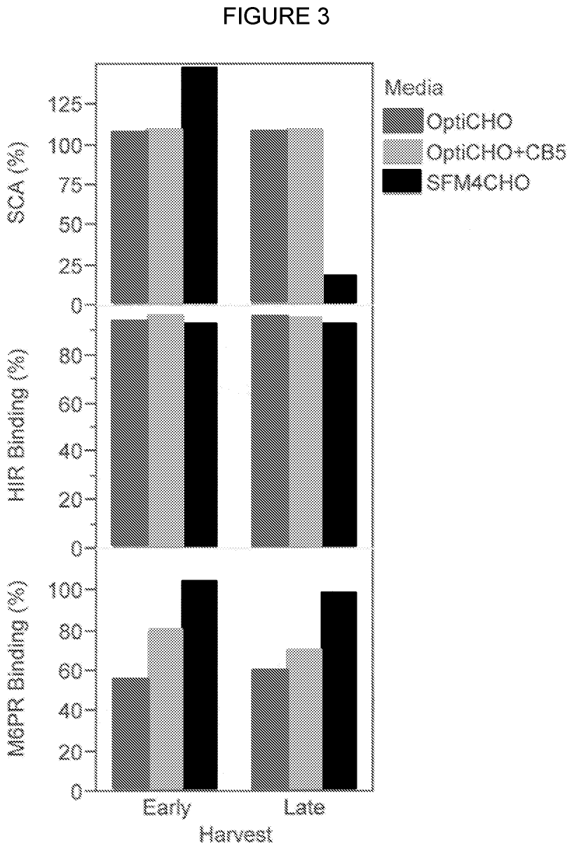

FIG. 3 demonstrates the Substrate Clearance assay (SCA), binding to the Human insulin receptor (% RS), and binding to M6P receptor (% RS).

Taken together, this example demonstrates that cell culture conditions containing chemically-defined OptiCHO media can be used to successfully modulate 2-M6P levels in HIRMab-I2S fusion protein and retain activity and substrate clearance to late harvests. This example demonstrates that product quality data confirms that OptiCHO media produces active material from early to late harvest, a substantial improvement over previous runs using media containing hydrolysates and animal components.

### Example 5: Analysis of Media on HIRMab-I2S Fusion Protein Glycan Map

The objective of this study was to assess media conditions and to characterize the glycan map of OptiCHO-produced HIRMab-I2S fusion protein product quality as compared to media conditions containing hydrolysates and animal components (SFM4CHO).

This study evaluated mannose-6-phosphate and sialic acid levels in HIRMab-I2S fusion protein obtained from a chemically defined medium bioreactor.

FIG. 4 demonstrates the levels of mannose-6-phosphate glycan content (1-M6P and 2-M6P) in early, mid, and late HIRMab-I2S fusion protein harvest materials obtained under aforementioned media conditions.

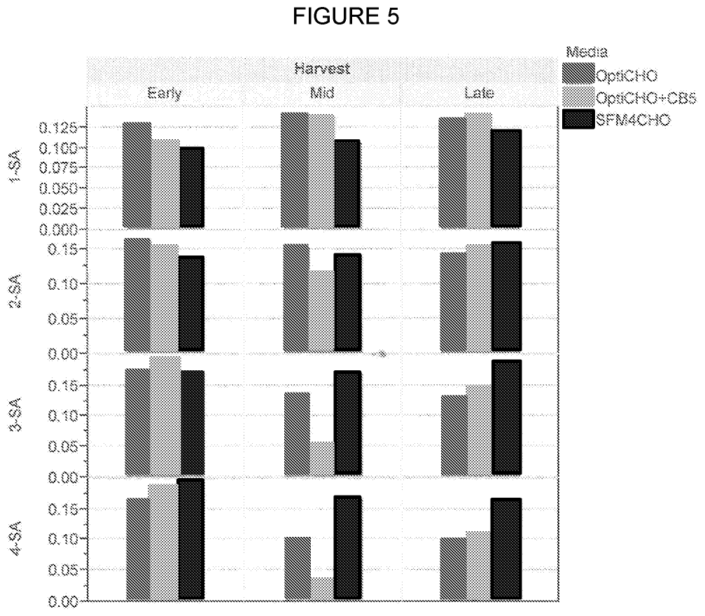

FIG. 5 demonstrates the levels of sialylation glycan content (1-, 2-, 3-, and 4-SA) in early, mid, and late HIRMab-I2S fusion protein harvest materials obtained under aforementioned media conditions.

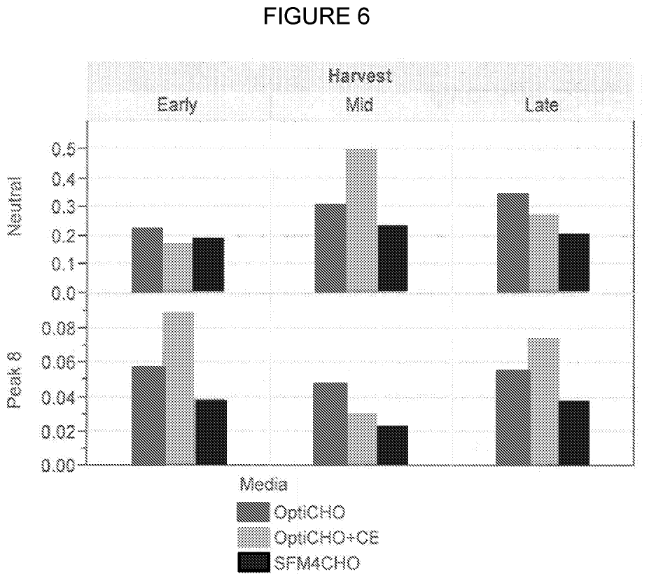

FIG. 6 demonstrates the levels of neutral and Peak 8 glycan content in early, mid, and late HIRMab-I2S fusion protein harvest materials obtained under aforementioned media conditions.

Typically, using a process described herein, OptiCHO media conditions produced lower 2-M6P levels than that in the SFM4CHO control. Taken together, this example demonstrates that cell culture conditions containing chemically-defined OptiCHO media can be used to successfully modulate 2-M6P levels in HIRMab-I2S fusion protein.

### Glycan Map—Mannose-6-Phosphate and Sialic Acid Content

HIRMab-I2S fusion protein harvest samples from early, middle, and late harvest stages were pooled and captured as described in Example 4. The glycan and sialic acid compositions of HIRMab-I2S fusion protein harvest materials were determined. Quantification of the glycan composition was performed, using anion exchange chromatography to produce a glycan map. As described below, the glycan map of I2S fusion protein purified under conditions described herein consists of seven peak groups, eluting according to an increasing amount of negative charges, at least partly derived from sialic acid and mannose-6-phosphate glycoforms resulting from enzymatic digest. Briefly, HIRMab-I2S fusion protein from OptiCHO, OptiCHO+Cell boost 5, and control SFM4CHO cell cultures were treated with either (1) purified neuraminidase enzyme (isolated from Arthrobacter Ureafaciens (10 mU/uL), Roche Biochemical (Indianapolis, Ind.), Cat. #269 611 (1U/100 μL)) for the removal of sialic acid residues, (2) alkaline phosphatase for 2 hours at 37±1° C. for complete release of mannose-6-phosphate residues, (3) alkaline phosphatase+neuraminidase, or (4) no treatment. Each enzymatic digest was analyzed by High Performance Anion Exchange Chromatography with Pulsed Amperometric Detection (HPAE-PAD) using a CarboPac PA1 Analytical Column equipped with a Dionex CarboPac PA1 Guard Column. A series of sialic acid and mannose-6-phosphate standards in the range of 0.4 to 2.0 nmoles were run for each assay. An isocratic method using 48 mM sodium acetate in 100 mM sodium hydroxide was run for a minimum of 15 minutes at a flow rate of 1.0 mL/min at ambient column temperature to elute each peak. As indicated in FIGS. 4, 5, and 6, the glycan map for HIRMab-I2S fusion protein from serum-free medium showed representative elution peaks (in the order of elution) constituting neutrals, mono-, disialyated, monophosphorylated, trisialyated and hybrid (monosialyated and capped mannose-6-phosphate), tetrasialylated and hybrid (disilaylated and capped mannose-6-phosphate) and diphosphorylated glycans.

### Example 6: HIR Mab-I2S Fusion Protein Capture and Purification Process

This example demonstrates a method to accurately measure specific activity and correct inhibitory effects for a HIR Mab-I2S fusion protein. An exemplary specific activity assay is depicted in FIG. 7.

IdoA2S-4-MU was custom synthesized by Carbosynth (Compton, Berkshire, UK) and α-L-iduronidase (IDUA) was generated by Shire. 4-Methyl-umbelliferone (4-MU) sodium salt was obtained from Sigma-Aldrich. Opti CHO culture medium was from ThermoFisher. I2S activity measurement of a HIR Mab-I2S fusion protein was carried out using a two-step plate based method with fluorescence detection as described by Voznyi Y V, Keulemans, J L M, van Diggelen, O P (2001) J. Inherit. Metab. Dis. 24 675-680 (herein incorporated in its entirety for all purposes) with slight modification. The first reaction catalyzed by a HIR Mab-I2S fusion protein was initiated by mixing equal volumes of substrate solution and the diluted in-process sample; the substrate IdoA2S-4-MU is hydrolyzed to umbelliferyl-α-L-idopyranosiduronic acid (IdoA-4-MU) and sulfate. In the second reaction, a complete conversion of IdoA-4-MU to 4-MU was achieved by the addition of excess amount of IDUA.

Typically, an IdoA2S-4-MU assay measures the ability of an I2S protein via a two-step method (FIG. 7). In the first step; the substrate IdoA2S-4-MU was hydrolyzed to umbelliferyl-α-L-idopyranosiduronic acid (IdoA-4-MU) and sulfate. In the second step, a complete conversion of IdoA-4-MU to naturally fluorescent 4-MU can be achieved by the addition of excess amount of IDUA. Typically, the mean fluorescence units (MFU) generated by I2S test samples with known activity can be used to generate a standard curve, which can be used to calculate the enzymatic activity of a sample of interest. Specific activity may then calculated by dividing the enzyme activity by the protein concentration.

In some embodiments, specific activity is measured using a plate-based fluorometric enzyme activity assay. In some embodiments, the reaction is carried out in 96-well PCR plate with a temperature controlled thermocycler. In such embodiments, the reaction can be initiated by mixing 20 μL each of 2 mM IdoA2S-4-MU substrate solution and 5 ng/mL I2S sample solution in 2× assay buffer, which are incubated for one hour at 37° C. In some embodiments, the buffer comprises a 50 mM acetate-buffered reaction mixture, pH 5.2, containing 0.03 mg/mL of BSA. In some embodiments, 40 μL of 25 μg/mL IDUA in McIlvaine's buffer (0.40 M sodium phosphate, 0.20 M citrate, 0.02% sodium azide, pH 4.5) can be added to arrest the I2S reaction, which can be incubated for an additional hour at the same temperature. In some embodiments, the second step reaction is quenched by addition of 200 μL of 0.5 M sodium carbonate solution, pH 10.7. In such embodiments, the observed fluorescence of 4-MU can be measured at λₑₓ and λₑₘ of 365 and 450 nm, respectively.

The above assay was be modified as necessary to understand matrix interference. The buffers tested for the matrix effect are shown in Table 1.

The reaction was carried out in the same way as the assay with a HIR Mab-I2S fusion protein sample except the final substrate IdoA2S-4-MU concentration was varied. The substrate solution was serially diluted, before mixing with a HIR Mab-I2S fusion protein, to give concentrations of 31.25 to 2000 μM in the final reaction mixture.

Kₘ was determined by fitting the dependence of the observed activity on the concentration of the substrate to the Michaelis-Menten equation as described in Equation 1 below:

$$\frac{v}{[ E ]} = + \frac{{{k}_{cat}}^{∞} [ S ]}{{K}_{m} + [ S ]} \qquad ( Equation 1 )$$

Assay in the presence of buffer matrix. For assessment of buffer matrix effect, a fixed amount of a HIR Mab-I2S fusion protein was mixed with various diluted in-process buffers. 10 μL each of the diluted buffer solution and the substrate solution were mixed with 20 μL of the enzyme solution in 2× assay buffer to start the 1st step reaction, after which the assay proceeded as described above. The final enzyme concentration in the reaction was 2.5 ng/mL.

Enzyme specific activity (v/[E]) vs. inverse of dilution factor (1/DF) were fitted to the equation for rapid equilibrium mixed inhibition as per Equation 2 using a value of Kₘ=386 μM determined from Michaelis-Menten analysis of a HIR Mab-I2S fusion protein as described above

$$\frac{v}{[ E ]} = \frac{{k}_{cat} S}{{K}_{m} ( 1 + \frac{[ \frac{C}{DF} ]}{{K}_{is}} ) + S ( 1 + \frac{[ \frac{C}{DF} ]}{{K}_{ii}} )} \qquad ( Equation 2 )$$

$$[ \frac{C}{DF} ]$$

where is the apparent concentration of the inhibitory components

Kᵢₛ is the inhibition constant for inhibitor binding to free enzyme

Kᵢᵢ is the inhibition constant for inhibitor binding to the ES complex

When the substrate concentration is fixed, the parameters Kᵢᵢ and Kᵢₛ become redundant, thus the same fit curve can be obtained regardless of whether the inhibition is competitive, noncompetitive, uncompetitive or mixed. Thus, once the necessary parameters were determined from the fit, the same inhibition-free activity (v₀/[E]) was calculated regardless of which inhibition modes are actually operating (Equation 3).

$$\frac{{v}_{0}}{[ E ]} = \frac{v}{[ E ]} ( \frac{{K}_{m} ( 1 + \frac{C}{{DFK}_{is}} ) + S ( 1 + \frac{C}{{DFK}_{ii}} )}{{K}_{m} + S} \\ where {v}_{0} = catalytic rate in the absence of inhibitory component \qquad ( Equation 3 )$$

Simplification of the data treatment by linearization of the rate Equation 2. The inhibition free activity can be also calculated using the linearized equation per Equation 4.

$$\begin{matrix}\frac{[ E ]}{v} = ( \frac{{K}_{m}}{{k}_{cat} [ S ] {K}_{is}} + \frac{[ S ]}{{k}_{cat} [ S ] {K}_{ii}} ) [ \frac{C}{DF} ] + \frac{{K}_{m} + [ S ]}{{k}_{cat} [ S ]} & ( Equation 4 ) \\ or & \\ \frac{[ E ]}{v} = \frac{a}{DF} + b & \\ where & \\ a = ( \frac{{K}_{m}}{{k}_{cat} [ S ] {K}_{is}} + \frac{[ S ]}{{k}_{cat} [ S ] {K}_{ii}} ) C & \\ and & \\ b = \frac{{K}_{m} + [ S ]}{{k}_{cat} [ S ]} = \frac{[ E ]}{{v}_{0}} & \end{matrix}$$

The above equation can be simplified to:

$$\frac{[ E ]}{v} = \frac{a}{DF} + \frac{[ E ]}{{v}_{0}} \qquad ( Equation 5 )$$

The activity in the absence of inhibitors was calculated from Equation 6 after fitting the data to the linear equation.

$$\begin{matrix}\frac{{v}_{0}}{[ E ]} = \frac{\frac{v}{[ E ]} DF}{DF - \frac{v}{[ E ]} a} & ( Equation 6 ) \\ where a = Slope of \frac{[ E ]}{v} vs . \frac{1}{DF} plot & \end{matrix}$$

However, in simple linear fitting of the reciprocal plot, smaller values of v/[E] with larger random errors are heavily weighted and could distort the results. To avoid this, an appropriate weighting factor was applied to the data points or extremely low values were simply omitted from the fit.

It was noticed that the measured specific activity for different in-process samples can depend on the concentration of the diluted sample used in the assay. In some embodiments, a higher concentration (lower dilution factor) of sample gave lower specific activity values. Further experiments to determine if this effect is due to inhibition by in-process sample buffer matrix were conducted as described below.

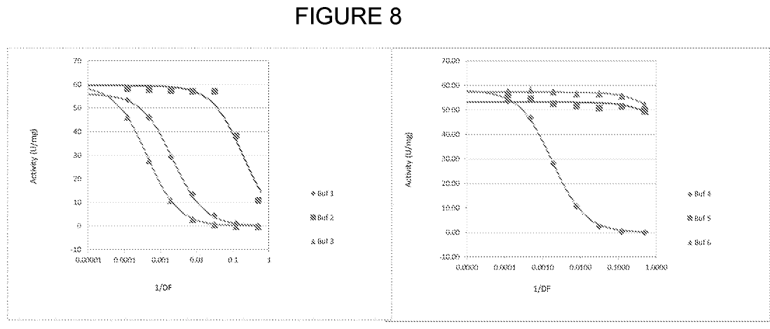

In order to assess the effect of the buffer matrix on the measured specific activity values, the assay was carried out at fixed concentration of a HIR Mab-I2S fusion protein in varying concentrations of six in process buffer solutions (Table 1). A decrease in specific activity at higher buffer concentration was observed with buffers 1, 2, 3, and 4 and a smaller decrease was observed with buffer 5 and 6. The results of the experiment are shown in FIG. 8.

TABLE 1
In Process Sample Buffers Tested for
Inhibition of Enzymatic Activity

| | |
|---|---|
| Buffer 1 | 50 mM Sodium Citrate, pH 3.6) + 3-vol (2M Tris base)] |
| | (Final pH 5.5) |
| Buffer 2 | 25 mM MES-Tris, 1.5M Sodium Chloride, pH 7.0 |
| Buffer 3 | Drug Substance Formulation Buffer [20 mM Sodium |
| | Phosphate, 140 mM Sodium Chloride, pH 6.0] |
| Buffer 4 | Opti CHO Media |
| Buffer 5 | 25 mM Tris, 25 mM Sodium Chloride, 5 mM EDTA, pH 7.1 |
| Buffer 6 | 25 mM MES, 150 mM Sodium Chloride, pH 5.5 |

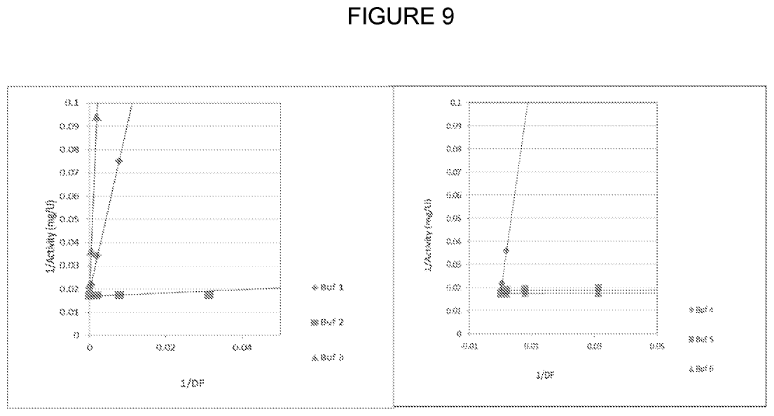

The values of matrix-free activity (v₀/[E]) and the parameters (Kᵢₛ/C, with Kᵢᵢ=∞) are determined from the fit of observed specific activity (v/[E]) vs. DF to equation (4) for each in process sample buffer. These results are shown in Table 2A. Once the parameters from the nonlinear fit are known, the matrix free activity v₀[E] can be determined using data from a single concentration point using the same equation. The values of inhibition free activity determined from single concentration points were consistent regardless of the dilution factor, except for the case of high inhibition (low specific activity) where relative random errors are larger. Matrix free activity can also be determined by fitting the data to the linearized Equation 5 if the low activity values are omitted due to high random error (FIG. 9). The calculated matrix-free activity values were comparable to those from the nonlinear fit (see Tables 2A and 2B).

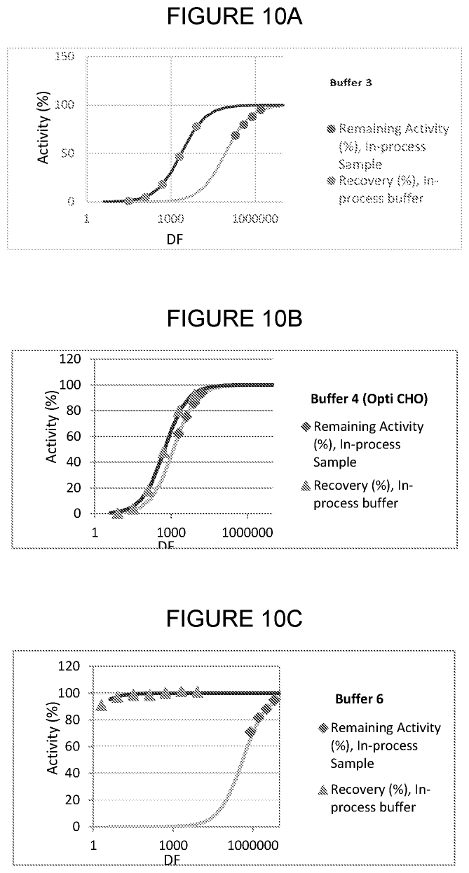

The data from this experiment for buffers 3, 4 and 6 was overlaid with the results of the corresponding real in-process sample serially diluted in water across the dilution range indicated (FIGS. 10A-10C). The results show that for buffers 3 and 6, inhibition is much stronger for the real in-process sample than for buffer alone (constant [E]). These results indicate that the observed inhibition in the real samples in buffers 3 and 6 is not due to buffer-matrix alone.

In some embodiments, sources of the additional inhibition observed in the real in-process samples, beyond what is attributable to buffer matrix alone, are:
(1) Substrate depletion; more substrate is consumed at smaller DF (higher enzyme concentration) thereby reducing v/[E] and/or
(2) Product Inhibition; at lower DF of in-process samples, enzyme concentration is higher thus generating more product, which causes greater inhibition.

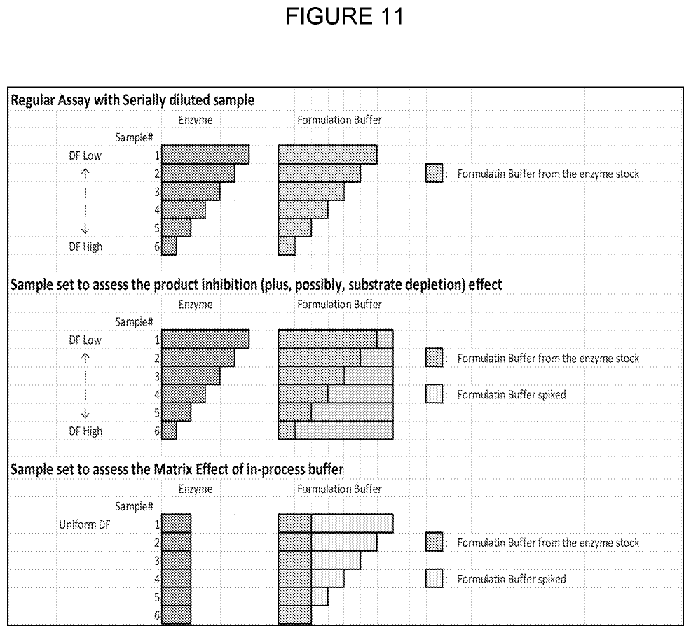

To investigate the source of inhibition effects, three sets of samples were created with buffer 3 (DS formulation buffer). Set A: varied enzyme concentration and varied buffer concentration, Set B: varied enzyme concentration and constant buffer concentration, and Set C: constant enzyme concentration and varied buffer concentration (FIG. 11).

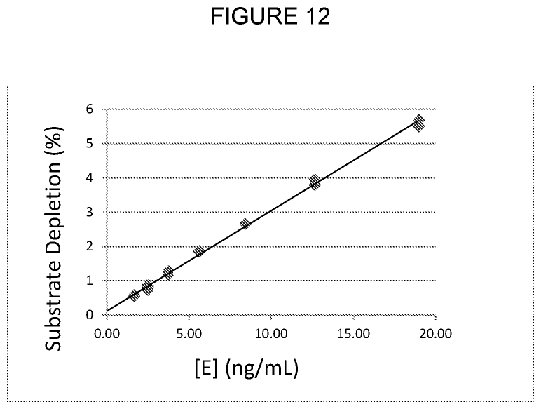

Substrate depletion was not the cause of the observed inhibition since the amount of substrate converted to product was <6%, so that [S]≈[S]ᵢₙᵢₜᵢₐₗ (FIG. 12).

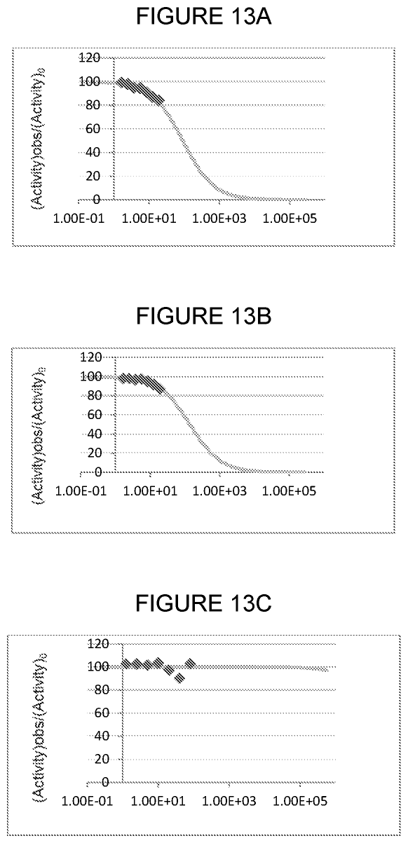

The data from sample Set A (varied [E], varied [buffer]) and Set B (varied [E], constant [Buffer]) indicate a stronger dependence of specific activity on concentration (proportional to 1/DF) than Set C (constant [E], varied [buffed]). These results (FIGS. 13A-13C) demonstrated the inhibition was not caused by matrix alone and was due to product inhibition.

These results indicate that both matrix and product can cause inhibition and decreased specific activity of a HIR Mab-I2S fusion protein at lower sample DF. For some in-process sample types, product inhibition is the dominant source of inhibition observed. The relative importance of matrix vs. product inhibition is determined by the nature of the matrix and dilution factor used in the assay.

The decreased specific activity at lower dilution factors of a HIR Mab-I2S fusion protein in in-process samples can be due to a combination of matrix inhibition and product inhibition. This was shown in separate experiments to demonstrate inhibition by matrix components (varying buffer matrix concentration, constant enzyme concentration) vs. inhibition by product (varying enzyme concentration, constant buffer matrix concentration).

The following method was used to determine inhibition-free specific activity of in-process samples when enzyme concentration is sufficiently high and matrix effects are not significant:
I. Determine dependence of specific activity on [E] using purified SHP631. Perform a linear fit of [E]/v vs. 1/DF to obtain the slope (a′ from Equation 4).
II. Measure the specific activity (v/[E]) of the in-process sample at a single defined dilution factor.
III. Calculate inhibition free specific activity (v₀/[E]) from Equation 6 using the slope a from step (i).

For in-process sample types where the source of inhibition has not been identified, inhibition-free specific activity can be determined by assaying at various dilutions, plotting [E]/v vs. 1/DF and extrapolating to infinite dilution (1/DF=0). This would allow direct comparison of enzyme activity values across various in process sample types.

What is claimed is:

1. A composition comprising a purified fusion protein including an immunoglobulin and an iduronate-2-sulfatase (I2S), wherein the fusion protein comprises at least about 60% conversion of the cysteine residue corresponding to Cys59 of SEQ ID NO:1 to Cα-formylglycine (FGly), wherein the purified fusion protein is characterized with between 1% and 10% 2-mannose-6-phosphate (2-M6P) peak area on glycan map.

2. The composition of claim 1, wherein the purified fusion protein is characterized with between 4% and 9% 2-mannose-6-phosphate (2-M6P) peak area on glycan map.

3. The composition of any one of the preceding claims, wherein the purified fusion protein is characterized with between 5.2% and 7.2% 2-mannose-6-phosphate (2-M6P) peak area on glycan map.

4. A composition comprising purified fusion protein including an immunoglobulin and an iduronate-2-sulfatase (I2S), wherein the fusion protein comprises at least about 60% conversion of the cysteine residue corresponding to Cys59 of SEQ ID NO:1 to Cα-formylglycine (FGly), wherein the purified fusion protein comprises between, on average, about 3.0 mol/mol and about 4.0 mol/mol mannose-6-phosphate (2-M6P) residues per molecule.

5. The composition of any one of the preceding claims, wherein the purified fusion protein comprises at least about 70% conversion of the cysteine residue corresponding to Cys59 of SEQ ID NO:1 to Cα-formylglycine (FGly).

6. The composition of any one of the preceding claims, wherein the purified fusion protein comprises at least about 80% conversion of the cysteine residue corresponding to Cys59 of SEQ ID NO:1 to Cα-formylglycine (FGly).

7. The composition of any one of the preceding claims, wherein the purified fusion protein comprises at least about 90% conversion of the cysteine residue corresponding to Cys59 of SEQ ID NO:1 to Cα-formylglycine (FGly).

8. The composition of any one of the preceding claims, wherein the purified fusion protein comprises at least about 95% conversion of the cysteine residue corresponding to Cys59 of SEQ ID NO:1 to Cα-formylglycine (FGly).

9. The composition of any one of the preceding claims, wherein the purified fusion protein comprises at least about 98% conversion of the cysteine residue corresponding to Cys59 of SEQ ID NO:1 to Cα-formylglycine (FGly).

10. The composition of any one of the preceding claims, wherein the purified fusion protein is derived from mammalian cells.

11. The composition of any one of the preceding claims, wherein the purified fusion protein is derived from CHO cells.

12. The composition of any one of the preceding claims, wherein the purified fusion protein comprises between 5.2% and 7.2% 2-M6P residue levels.

13. The composition of any one of the preceding claims, wherein the purified fusion protein includes an immunoglobulin comprising a chimeric monoclonal antibody.

14. The composition of any one of the preceding claims, wherein the immunoglobulin comprises a chimeric monoclonal antibody that binds to Human Insulin Receptor (HIR).

15. The composition of any one of the preceding claims, wherein the purified fusion protein comprises a human insulin receptor monoclonal antibody fused with I2S.

16. The composition of claim 15, wherein the purified fusion protein comprises an amino acid sequence at least 80%, 85%, 90% or 95% identical to SEQ ID NO: 11.

17. The composition of claim 16, wherein the purified fusion protein comprises an amino acid sequence identical to SEQ ID NO: 11.

18. The composition of any one of claims 14-17, wherein the chimeric monoclonal antibody comprises a recombinant human IgG light chain.

19. The composition of claim 18, wherein the recombinant human IgG light chain comprises an amino acid sequence at least 80%, 85%, 90% or 95% identical to SEQ ID NO: 12.

20. The composition of claim 19, wherein the recombinant human IgG light chain comprises an amino acid sequence identical to SEQ ID NO: 12.

21. The composition of any one of the preceding claims, wherein the fusion protein does not comprise a linker.

22. The composition of any one of the preceding claims, wherein the Human Insulin Receptors mediate transport via endogenous brain capillary endothelial insulin receptors.

23. The composition of any one of the preceding claims, wherein the Human Insulin Receptors mediate transport via endogenous neuronal insulin receptors.

24. The composition of any one of the preceding claims, wherein the purified fusion protein includes an iduronate-2-sulfatase comprising 2-M6P residues.

25. The composition of any one of the preceding claims, wherein the 2-M6P residues bind to M6P receptors.

26. The composition of any one of the preceding claims, wherein the 2-M6P receptors mediate transport via endogenous lysosomal M6P receptors.

27. The composition of any one of the preceding claims, wherein the 2-M6P residues facilitate at least about 60% binding to M6P receptors.

28. The composition of any one of the preceding claims, wherein the 2-M6P residues facilitate at least about 70% binding to M6P receptors.

29. The composition of any one of the preceding claims, wherein the 2-M6P residues facilitate at least about 75% binding to M6P receptors.

30. The composition of any one of the preceding claims, wherein the immunoglobulin facilitates at least about 70% binding to Human Insulin Receptors.

31. The composition of any one of the preceding claims, wherein the immunoglobulin facilitates at least about 80% binding to Human Insulin Receptors.

32. The composition of any one of the preceding claims, wherein the immunoglobulin facilitates at least about 90% binding to Human Insulin Receptors.

33. The composition of any one of the preceding claims, wherein the immunoglobulin facilitates at least about 95% binding to Human Insulin Receptors.

34. The composition of any one of the preceding claims, wherein the purified fusion protein has a specific activity of at least about 3 U/mg as determined by a plate-based fluorometric enzyme assay.

35. The composition of any one of the preceding claims, wherein the purified fusion protein contains less than 10 ng/mg (ppm) HCP.

36. The composition of any one of the preceding claims, wherein the purified fusion protein contains at least 15 mol/mol sialic acid content.

37. The composition of any one of the preceding claims, wherein the purified fusion protein contains at least 20 mol/mol sialic acid content.

38. A method comprising purifying a fusion protein including an immunoglobulin and an iduronate-2-sulfatase (I2S) from an impure preparation by conducting one or more of affinity chromatography, cation-exchange chromatography, and multimodal chromatography.

39. The method of claim 38, wherein the affinity chromatography is Protein A Antibody chromatography.

40. The method of claim 38, wherein the cation-exchange chromatography is Capto SP ImpRes chromatography.

41. The method of claim 38, wherein the multimodal chromatography is Capto Adhere chromatography.

42. The method of claim 38, wherein the method involves 3 chromatography steps.

43. The method of claim 38, wherein the method conducts the affinity chromatography, cation-exchange chromatography, and multimodal chromatography in that order.

44. The method of claim 38, wherein the affinity chromatography column is eluted using an elution buffer comprising an isocratic sodium citrate elution.

45. The method of claim 38, wherein the isocratic sodium citrate elution comprises a range from 10-100 mM sodium citrate.

46. The method of claim 38, wherein the affinity chromatography column is run at a pH of between 3.3 and 3.9.

47. The method of claim 38, wherein the cation-exchange chromatography column is eluted using an elution buffer comprising an isocratic NaCl elution.

48. The method of claim 47, wherein the NaCl elution comprises a range from 10-300 mM NaCl.

49. The method of claim 48, wherein the cation-exchange chromatography column is run at a pH of between 5.2 and 5.8.

50. The method of claim 38, wherein the multimodal chromatography column is operated in flow through mode and/or bind/elute mode.

51. The method of any one of the preceding claims, wherein a salt concentration of between 1.0 and 2.0 M NaCl is used in loading and washing the chromatography columns.

52. The method of claim 38, wherein the multimodal chromatography column is run at a pH of about 7.0.

53. The method of any one of claims 38-52, wherein the method further comprises a step of viral inactivation.

54. The method of any one of claims 38-53, wherein the method further comprises a step of vial filtration after the last chromatography column.

55. The method of any one of claims 38-54, wherein the fusion protein including an immunoglobulin and an I2S protein is produced by mammalian cells cultured in chemically defined medium.

56. The method of any one of claims 38-54, wherein the mammalian cells are CHO cells.

57. The method of any one of the preceding claims, wherein the mammalian cells are cultured in a bioreactor.

58. The method of any one of claims 38-54, wherein the bioreactor operates as a stirred tank perfusion bioreactor process.

59. The method of any one of claims 38-54, wherein the impure preparation is prepared from the chemically defined medium containing fusion protein secreted from the mammalian cells.

60. A pharmaceutical composition comprising a purified fusion protein including an immunoglobulin and an I2S protein purified according to a method of any one of the preceding claims.

61. The pharmaceutical composition of claim 60, wherein the immunoglobulin comprises a chimeric monoclonal antibody that binds to the Human Insulin Receptor (HIR).

62. The pharmaceutical composition of claim 61, wherein the purified fusion protein comprises an I2S polypeptide and a chimeric monoclonal antibody that binds to the Human Insulin Receptor (HIR).

63. The pharmaceutical composition of claim 62, wherein the purified fusion protein comprises an amino acid sequence at least 80%, 85%, 90% or 95% identical to SEQ ID NO: 11.

64. The pharmaceutical composition of claim 62, wherein the purified fusion protein comprises an amino acid sequence identical to SEQ ID NO: 11.

65. The pharmaceutical composition of any one of claims 62-64, wherein the chimeric monoclonal antibody comprises a recombinant human IgG light chain.

66. The pharmaceutical composition of claim 65, wherein the recombinant human IgG light chain comprises an amino acid sequence at least 80%, 85%, 90% or 95% identical to SEQ ID NO: 12.

67. The pharmaceutical composition of claim 66, wherein the recombinant human IgG light chain comprises an amino acid sequence identical to SEQ ID NO: 12.

68. A method of treating Hunter syndrome comprising administering to a subject in need of treatment a pharmaceutical composition of any one of claims 60-67.


SEQUENCE LISTING (12 sequences)

SEQ ID NO: 1 - protein, 525 aa, Human
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - protein, 550 aa, Human
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 3 - protein, 312 aa, Human
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQEDQSSTGFRLKTSSTRKYK

SEQ ID NO: 4 - protein, 343 aa, Human
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGFLMRTNT

SEQ ID NO: 5 - protein, 989 aa, Human [sequence omitted]

SEQ ID NO: 6 - protein, 234 aa, Human
METPAQLLFLLLLWLPDTTGDIQMTQSPSSLSASLGERVSLTCRASQDIGGNLYWLQQGPDGTIKRLIYATSSLDSGVPKRFSGSRSGSDYSLTISSLESEDFVDYYCLQYSSSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 13 aa, Human
GGGGGAAAAGGGG

SEQ ID NO: 8 - protein, 3 aa, Human
GAP

SEQ ID NO: 9 - protein, 6 aa, Human
GGGGGP

SEQ ID NO: 10 - protein, 51 aa, Human
GAPGGGGGAAAAGGGGGAPGGGGGAAAAGGGGGAPGGGGGAAAAGGGGGAP

SEQ ID NO: 11 - protein, 989 aa, Human [sequence omitted]

SEQ ID NO: 12 - protein, 234 aa, Human; identical to SEQ ID NO: 6